(19) United States
(12) Patent Application Publication
(10) Pub. No.: US 2025/0066704 A1
(43) Pub. Date: Feb. 27, 2025

(54) CIRCUIT LADDER MICROFLUIDIC PLATFORM (CLAMP) FOR RAPID ANTIMICROBIAL SUSCEPTIBILITY TESTING (AST)

(71) Applicant: CORNELL UNIVERSITY, Ithaca, NY (US)

(72) Inventors: Alireza ABBASPOURRAD, Ithaca, NY (US); Ann NGUYEN, Ithaca, NY (US); Mohammad YAGHOOBI, Ithaca, NY (US); Morteza AZIZI, Ithaca, NY (US)

(21) Appl. No.: 18/812,562

(22) Filed: Aug. 22, 2024

Related U.S. Application Data
(60) Provisional application No. 63/578,002, filed on Aug. 22, 2023.

(51) Int. Cl. C12M 3/06; C12M 1/00; C12M 1/02; C12M 1/34; C12Q 1/18; G01N 21/64

(52) U.S. Cl. CPC C12M 23/16; C12M 27/20; C12M 29/14; C12M 41/22; C12M 41/46; C12Q 1/18; G01N 21/6486

(57) ABSTRACT

The present disclosure relates to a microfluidic circuit comprising a drug inlet port; an outlet port; a drug inlet main channel fluidically connecting the drug inlet port and the outlet port, where said drug inlet main channel comprises (i) a plurality of serpentine mixers and (ii) a plurality of dead-end first microchamber sets; a negative inlet port; a negative inlet main channel fluidically connecting the negative inlet port and the drug inlet main channel; a plurality (n) of ladder channels, where each of the plurality (n) of the ladder channels is fluidically connected to both the drug inlet main channel and the negative inlet main channel; and an outlet channel fluidically connected to the drug inlet main channel between the drug inlet port and the outlet port. Also disclosed is a microfluidic device comprising a microfluidic circuit of the present disclosure and a method for performing an assay.

This application claims the benefit of U.S. Provisional Patent Application Ser. No. 63/578,002, filed Aug. 22, 2023, which is hereby incorporated by reference in its entirety.

This invention was made with government support under 2019-38420-28975 awarded by the United States Department of Agriculture's National Institute of Food and Agriculture. The government has certain rights in the invention.

### FIELD

This disclosure relates to a microfluidic device and methods for performing a microfluidic assay.

### BACKGROUND

Antibiotic resistance (AR) is a rising crisis worldwide. The rise of AR has largely been attributed to the overuse of antibiotics in human and veterinary medicine, and animal husbandry (Schoepp et al., “Rapid Pathogen-Specific Phenotypic Antibiotic Susceptibility Testing using Digital LAMP Quantification in Clinical Samples,” Sci. Transl. Med. 9, eaal3693 (2017) and Hong et al., “Assessing Antibiotic Permeability of Gram-Negative Bacteria via Nanofluidics,” ACS Nano 11:6959-6967 (2017)). AR can be caused by a number of factors including over-prescribing of antibiotics, a lack of rapid laboratory tests to help identify antibiotic susceptibility, and prescribing either an ineffective or only marginally effective antibiotic (US Food and Drug Administration. Antimicrobial Resistance Information from FDA (FDA, 2022)). Therefore, advancing the development and use of rapid diagnostic tests to identify and characterize resistant bacteria, leading to targeted antibiotic administration is a key in combating antibiotic resistance.

Currently, the workflow of a bacterial infection diagnosis takes 2-3 days and includes three steps: isolation, identification, and sensitivity (Reller et al., “Antimicrobial Susceptibility Testing: A Review of General Principles and Contemporary Practices,” Clin. Infect. Dis. 49:1749-1755 (2009); Clinical and Laboratory Standards Institute. Performance Standards for Antimicrobial Susceptibility Testing. 32nd. CLSI Supplement (Clinical and Laboratory Standards Institute, 2022); and US Food and Drug Administration. Class II Special Controls Guidance Document: Antimicrobial Susceptibility Test (AST) Systems (U.S. Department of Health and Human Services, 2009)). It starts with sample collection, and the sample is either processed on location or shipped to an accredited diagnostic lab for sensitivity test. Upon arrival, the sample is plated onto non-selective and/or selective agar to enrich or isolate the bacteria in the sample. Then the infective agent is identified by morphological, biochemical, or molecular assays. This process can take up to 2 days depending on the type of sample, bacteria, and techniques used at each clinical microbiology lab. Then, after the bacteria are isolated, antibiotic susceptibility testing (AST) is conducted, which takes another 16-20 hours.

The following two phenotypic cultures are the current standard methods for AST: agar disk diffusion methods (Alagumaruthanayagam et al., “Evaluation of Solid (Disc Diffusion)—and Liquid (Turbidity)—Phase Antibiogram Methods for Clinical Isolates of Diarrheagenic E. coli and Correlation with Efflux,” J. Antibiot. (Tokyo) 62:377-384 (2009) and Gefen et al., “TDtest: Easy Detection of Bacterial Tolerance and Persistence in Clinical Isolates by a Modified Disk-Diffusion Assay,” Sci. Rep. 7:41284 (2017)), which provide susceptibility results based on clearing-zone diameter, and Broth microdilution (BMD) methods (Wiegand et al., “Agar and Broth Dilution Methods to Determine the Minimal Inhibitory Concentration (MIC) of Antimicrobial Substances,” Nat. Protoc. 3:163 (2008)), which provide results based on culture turbidity or metabolic activity. Interpretive standard breakpoints are used to evaluate the growth of bacteria in the presence of antibiotics and to determine the pathogenic resistance. Because of the long time from collection of samples to receipt of the results of the culturing, there is often a lack of timely information about antibacterial resistance at the decision making stage of veterinary treatments, especially in emergency cases (Rodriguez & Taban, “A State-of-Art Review on Multi-Drug Resistant (MDR) Pathogens in Foods of Animal Origin: Risk Factors and Mitigation Strategies,” Front. Microbiol. 10:2091 (2019); Verraes et al., “Antimicrobial Resistance in the Food Chain: A Review,” Int. J. Environ. Res. Public. Health 10:2643-2669 (2013); and NAHMS. National Animal Health Monitoring System (NAHMS) Dairy 2007: Salmonella and Campylobacter on US Dairy Operations (https://allie.dbcls.jp/pair/NAHMS; National+Animal+Health+Monitoring+System.html) (2009)). Physicians and veterinarians treat based on clinical presentation of the infection using antibiotics with a broad spectrum of activity rather than wait for AST results to choose an antibiotic specific to the infection (Mohan et al., “A Multiplexed Microfluidic Platform for Rapid Antibiotic Susceptibility Testing,” Biosens. Bioelectron. 49:118-125 (2013) and Nguyen et al., “Diffusion-Convection Hybrid Microfluidic Platform for Rapid Antibiotic Susceptibility Testing,” Anal. Chem. 93:5789-5796 (2021)). Thus, there is a need to develop new platforms for rapid, inexpensive, and easily implemented AST approaches to reduce the assay time without compromising accuracy so that more targeted antibiotics can be used.

Recently, microfluidic platforms have been shown to improve the sensitivity and speed of AST. These platforms use techniques such as single cell confinement (Choi et al., “Direct, Rapid Antimicrobial Susceptibility Test from Positive Blood Cultures Based on Microscopic Imaging Analysis,” Sci. Rep. 7:1-13. (2017); Choi et al., “Rapid Antibiotic Susceptibility Testing by Tracking Single Cell Growth in a Microfluidic Agarose Channel System,” Lab. Chip 13:280-287 (2013); Baltekin et al., “Antibiotic Susceptibility Testing in less than 30 min Using Direct Single-Cell Imaging,” Proc. Natl. Acad. Sci. USA 114:9170-9175 (2017); Choi et al., “A Rapid Antimicrobial Susceptibility Test Based on Single-Cell Morphological Analysis,” Sci. Transl. Med. 6:267ra174-267ra174 (2014); Syal et al., “Antimicrobial Susceptibility Test with Plasmonic Imaging and Tracking of Single Bacterial Motions on Nanometer Scale,” ACS Nano 10:845-852 (2016); Li et al., “Gradient Microfluidics Enables Rapid Bacterial Growth Inhibition Testing,” Anal. Chem. 86:3131-3137 (2014); Li et al., “Adaptable Microfluidic System for Single-Cell Pathogen Classification and Antimicrobial Susceptibility Testing,” Proc. Natl. Acad. Sci. USA 116, 10270-10279 (2019)), microchamber arrays (Nguyen et al., “Diffusion-Convection Hybrid Microfluidic Platform for Rapid Antibiotic Susceptibility Testing,” Anal. Chem. 93:5789-5796 (2021); Weibull et al., “Bacterial Nanoscale Cultures for Phenotypic Multiplexed Antibiotic Susceptibility Testing,” J. Clin. Microbiol. 52:3310-3317 (2014); Azizi et al., “Nanoliter-Sized Microchamber/Microarray Microfluidic Platform for Antibiotic Susceptibility Testing,” Anal. Chem. 90:14137-14144 (2018); Azizi et al., “Gradient-Based Microfluidic Platform for One Single Rapid Antimicrobial Susceptibility Testing,” ACS Sens 6:1560-1571 (2021); Azizi et al., “Biological Small-Molecule Assays Using Gradient-Based Microfluidics,” Biosens. Bioelectron. 178:113038 (2021); Azizi et al., “Antimicrobial Susceptibility Testing in a Rapid Single Test via an Egg-Like Multivolume Microchamber-Based Microfluidic Platform,” ACS Appl. Mater. Interfaces 13:19581-19592 (2021); Lin et al., “An Antibiotic Concentration Gradient Microfluidic Device Integrating Surface-Enhanced Raman Spectroscopy for Multiplex Antimicrobial Susceptibility Testing,” Lab. Chip 22:1805-1814 (2022); and Yi et al., “Direct Antimicrobial Susceptibility Testing of Bloodstream Infection on SlipChip,” Biosens. Bioelectron. 135:200-207 (2019)), droplet microfluidics (Boedicker et al., “Detecting Bacteria and Determining their Susceptibility to Antibiotics by Stochastic Confinement in Nanoliter Droplets Using Plug-Based Microfluidics,” Lab. Chip 8:1265-1272 (2008); Churski et al., “Rapid Screening of Antibiotic Toxicity in an Automated Microdroplet System,” Lab. Chip 12:1629-1637 (2012); and Svensson et al., “Coding of Experimental Conditions in Microfluidic Droplet Assays Using Colored Beads and Machine Learning Supported Image Analysis,” Small 15:1802384 (2019)), and asynchronous magnetic bead rotation (Sinn et al., “Asynchronous Magnetic Bead Rotation (AMBR) Biosensor in Microfluidic Droplets for Rapid Bacterial Growth and Susceptibility Measurements,” Lab. Chip 11:2604-2611 (2011)) to immobilize or contain the bacteria and conduct AST. Microchamber-based platforms allow integration of microfluidic concentration gradient generators, thus offering high-throughput screening of multiple antibiotic concentrations and experimental controls on the same chip. Previous platforms using simple diffusion have produced continuous concentration gradients of antibiotics (Kim et al., “Microfluidic-Based Observation of Local Bacterial Density Under Antimicrobial Concentration Gradient for Rapid Antibiotic Susceptibility Testing,” Biomicrofluidics 13:014108 (2019) and Kim et al., “On-Chip Phenotypic Investigation of Combinatory Antibiotic Effects by Generating Orthogonal Concentration Gradients,” Lab. Chip 19:959-973 (2019)), but these approaches present a problem in determining the minimum inhibitory concentration (MIC) precisely. More recent platforms were successful at generating concentration gradients that are linear (Kim et al., “Microfluidic-Based Observation of Local Bacterial Density Under Antimicrobial Concentration Gradient for Rapid Antibiotic Susceptibility Testing,” Biomicrofluidics 13:014108 (2019); Kim et al., “On-Chip Phenotypic Investigation of Combinatory Antibiotic Effects by Generating Orthogonal Concentration Gradients,” Lab. Chip 19:959-973 (2019); Chen et al., “High-Throughput Generation of a Concentration Gradient on open Arrays by Serial and Parallel Dilution for Drug Testing and Screening,” Sens. Actuators B Chem 305:127487 (2020); Tang et al., “A Linear Concentration Gradient Generator based on Multi-Layered Centrifugal Microfluidics and its Application in Antimicrobial Susceptibility Testing,” Lab. Chip 18:1452-1460 (2018); Nguyen et al., “A High-Throughput Integrated Biofilm-on-a-Chip Platform for the Investigation of Combinatory Physicochemical Responses to Chemical and Fluid Shear Stress,” PloS One 17:e0272294 (2022)), logarithmic (Chen et al., “High-Throughput Generation of a Concentration Gradient on open Arrays by Serial and Parallel Dilution for Drug Testing and Screening,” Sens. Actuators B Chem 305:127487 (2020)), or sigmoidal (Avesar et al., “Nanoliter Cell Culture Array with Tunable Chemical Gradients,” Anal. Chem. 90:7480-7488 (2018)). Nevertheless, these techniques employ unstandardized concentration gradients. They deviate from the recommended standard quantitative AST technique that is based on 2-fold serial dilutions of an antibiotic to produce an exponential gradient (Clinical and Laboratory Standards Institute, “Performance Standards for Antimicrobial Susceptibility Testing,” 32nd. CLSI Supplement (Clinical and Laboratory Standards Institute, 2022)). Note that the susceptibility/resistance determination is based on the MIC interpretive breakpoints; the lack of harmonization between these methods and the current standards may present a challenge in interpreting results and consequently the utilization of these platforms.

The present disclosure is directed to overcoming these and other deficiencies in the art.

### SUMMARY

One aspect of the present disclosure relates to a microfluidic circuit comprising a drug inlet port; an outlet port; a drug inlet main channel fluidically connecting the drug inlet port and the outlet port, where said drug inlet main channel comprises (i) a plurality of serpentine mixers and (ii) a plurality of dead-end first microchamber sets; a negative inlet port; a negative inlet main channel fluidically connecting the negative inlet port and the drug inlet main channel; a plurality (n) of ladder channels, where each of the plurality (n) of the ladder channels is fluidically connected to both the drug inlet main channel and the negative inlet main channel; and an outlet channel fluidically connected to the drug inlet main channel between the drug inlet port and the outlet port.

Another aspect of the present disclosure relates to a microfluidic device comprising a support layer; a substrate layer disposed on the support layer; and a microfluidic circuit of the present disclosure, where the microfluidic circuit is disposed within the substrate layer.

A further aspect of the present disclosure relates to a method for performing an assay. This method involves loading a test solution into a microfluidic device of the present disclosure. A culture medium containing an antibiotic solution is loaded into the drug inlet port, a culture medium is loaded into the negative medium inlet port, and the device is then washed with oil to isolate each of the plurality of microchambers. The method further involves detecting a fluorescent signal in each of the plurality of microchambers.

The Examples of the present disclosure (infra) demonstrate the design and development of certain embodiments of a microfluidic system with an optimized ladder shaped network that combines and distributes culture medium and antibiotic in a 2-fold serial dilution. Based on previously established protocols for microscale culture-based AST (Nguyen et al., “Diffusion-Convection Hybrid Microfluidic Platform for Rapid Antibiotic Susceptibility Testing,” Anal. Chem. 93:5789-5796 (2021); Azizi et al., “Nanoliter-Sized Microchamber/Microarray Microfluidic Platform for Antibiotic Susceptibility Testing,” Anal. Chem. 90:14137-14144 (2018); Azizi et al., “Gradient-Based Microfluidic Platform for One Single Rapid Antimicrobial Susceptibility Testing,” ACS Sens 6:1560-1571 (2021); Azizi et al., “Biological Small-Molecule Assays Using Gradient-Based Microfluidics,” Biosens. Bioelectron. 178:113038 (2021); and Azizi et al., “Antimicrobial Susceptibility Testing in a Rapid Single Test via an Egg-Like Multivolume Microchamber-Based Microfluidic Platform,” ACS Appl. Mater. Interfaces 13:19581-19592 (2021), which are hereby incorporated by reference in their entirety), antibiotic susceptibility can be determined in less than 5 hours using the disclosed ladder system. The ability of this platform to perform AST of bacterial isolates as called for by the standard method was evaluated and the feasibility of its use on bacteria retrieved directly from canine urine samples without prior isolation or enrichment was investigated. This new platform provides an adaptable diagnostic tool and maintains relevance with current national and international interpretive standards.

### BRIEF DESCRIPTION OF THE DRAWINGS

FIG. 1 is a top view of one embodiment of a microfluidic circuit 10 of the present disclosure comprising a drug inlet port 12; an outlet port 22; a drug inlet main channel 16 fluidically connecting the drug inlet port 12 and the outlet port 14, where said drug inlet main channel 16 comprises (i) a plurality of serpentine mixers 18 and (ii) a plurality of dead-end first microchamber sets 20; a negative inlet port 22; a negative inlet main channel 24 fluidically connecting the negative inlet port 22 and the drug inlet main channel 16; a plurality of ladder channels, where each of the plurality of the ladder channels is fluidically connected to both the drug inlet main channel 16 and the negative inlet main channel 24; and an outlet channel 28 fluidically connected to the drug inlet main channel 16 between the drug inlet port and the outlet port 14, where the outlet channel comprises a plurality of mixers 48.

FIG. 2 is a top view of one embodiment of a microfluidic circuit 10 of the present disclosure comprising a drug inlet port 12; an outlet port 22; a drug inlet main channel 16 fluidically connecting the drug inlet port 12 and the outlet port 14, where said drug inlet main channel 16 comprises (i) a plurality of serpentine mixers 18 and (ii) a plurality of dead-end first microchamber sets 20; a negative inlet port 22; a negative inlet main channel 24 fluidically connecting the negative inlet port 22 and the drug inlet main channel 16, where the negative inlet main channel 16 is fluidically connected to a plurality of dead-end second microchamber sets 36; eight ladder channels, where each of the ladder channels is fluidically connected to both the drug inlet main channel 16 and the negative inlet main channel 24 and where seven ladder channels comprise a hydraulic resistor pinch 30; and an outlet channel 28 fluidically connected to the drug inlet main channel 16 between the drug inlet port and the outlet port 14, where the outlet channel comprises a plurality of mixers 48. Insets show enlarged views of an exemplary serpentine mixer 18 and each of the seven hydraulic resistor pinches 30.

FIG. 3 is a top view of a portion of an embodiment of a microfluidic circuit 10 of the present disclosure comprising a drug inlet main channel 16 comprising (i) two serpentine mixers 18 and (ii) two dead-end first microchamber sets 20 each comprising three distinct microchambers 44 fluidically connected to the drug inlet main channel 16 by side channels 32; a negative inlet main channel 24 comprising two dead-end second microchamber sets 36 fluidically connected to the negative inlet main channel 24 by side channels 38; and two ladder channels, where each of the ladder channels is fluidically connected to both the drug inlet main channel 16 and the negative inlet main channel 24 and where each ladder channel comprises a hydraulic resistor pinch 30. Also shown is an outlet channel 28 fluidically connected to the drug inlet main channel 16, where the outlet channel comprises a plurality of mixers 48.

FIG. 4 is a perspective view of one embodiment of a microfluidic device 50 of the present disclosure comprising microfluidic circuit 10 comprising a drug inlet port 12; an outlet port 22; a drug inlet main channel 16 fluidically connecting the drug inlet port 12 and the outlet port 14, where said drug inlet main channel 16 comprises (i) a plurality of serpentine mixers 18 and (ii) a plurality of dead-end first microchamber sets 20; a negative inlet port 22; a negative inlet main channel 24 fluidically connecting the negative inlet port 22 and the drug inlet main channel 16; a plurality of ladder channels, where each of the plurality of the ladder channels is fluidically connected to both the drug inlet main channel 16 and the negative inlet main channel 24; and an outlet channel 28 fluidically connected to the drug inlet main channel 16 between the drug inlet port and the outlet port 14. The microfluidic device further comprises a support layer 52 and substrate layer 54, where microfluidic circuit 10 is disposed on support later 52.

FIGS. 5A-5C are schematic representations of embodiments of microfluidic circuit and device design and principle of operation. FIG. 5A is a schematic representation of an embodiment of a ladder microfluidic system including an on-chip water bath. Insets showing the key features of the device: (i) the microchamber triplicates, (ii) the modified serpentine mixer, and (iii) the hydraulic resistor pinch on the side channels. FIG. 5B is a schematic representation of an embodiment of a protocol for platform operation: (i) device is loaded with bacteria suspension (blue), then (ii) loaded with culture medium containing antibiotic (red) and culture medium alone (blue) from two different inlets. The LCGG automatically generates a stable exponential decay concentration gradient of antibiotic, which diffuses into the microchambers. Finally, (iii) the channels are washed with oil (yellow), after a certain loading time, to isolate the microchambers. FIG. 5C is a schematic representation showing fluorescent intensities of resazurin in selected microchambers (M1-M9; PC=positive growth control) changes after 4-5 hours of incubation at 37° C. indicating bacteria metabolism. Three scenarios are possible where the test shows (i) a minimum inhibitory concentration (MIC) for the bacteria/antibiotic combination, (ii) resistance or MIC higher than the testing range, and (iii) invalid result due to no growth of bacteria.

FIGS. 6A-6F provide a computational fluid dynamics simulation and pressure nodes network of an embodiment of a microfluidic circuit of the present disclosure. FIG. 6A is shows flow distribution in the ladder. In the inset, unique numbers are assigned to the pressure nodes before and after each resistor feature, and at the junctions. The arrows show the direction of the flow. “i” and “k” are counters for the resistors and nodes, respectively. FIG. 6B shows the contours of pressure for constriction of (i) L=420 μm (ii) L=1650 μm (iii) L=4950 μm and (iv) the standard mixer resistors for the flow rate of 130 μL h⁻¹ for each of the channels. FIG. 6C is a graph showing the pressure at the inlet of the features in FIG. 6A. FIG. 6D is a graph showing hydraulic resistance of the constriction, as a function of the length for width of 40 μm. Simulations for seven constrictions are shown (L=420 μm, 1649 μm, 4070 μm, 4949 μm, 7990 μm, 10795 μm). At L=655 μm, the hydraulic resistance of the constriction is equal to that of the mixer. FIG. 6E is a graph showing that the ratio of the flow rates of the drug and the diluent at their respective inlets will affect the concentration profile of the microchambers. These results are from network simulations. FIG. 6F is a graph showing the dilution factor in the Concentration(%)=50×E1−i needs to be E=2, where i is the chamber number. The formula is fitted to the results in the part E to solve for E using least square algorithm.

FIGS. 7A-7E characterize a concentration gradient. FIG. 7A show images of an embodiment of microchambers showing the process of molecules diffusing into the microchambers from the main channel at t=0 s and t=3 min, and after the main channel is washed with oil. FIG. 7B is a concentration profile showing the average concentration of resazurin diffused into the microchambers over time, represented as the area under the curve of the concentration profile at each time point (n=5). FIG. 7C shows the concentration profile formed in ten microchambers of the ladder microfluidic system after a loading time of three min (n=3). FIG. 7D shows the linear correlation between the concentration profiles from computer simulation, of resazurin, fluorescein, and Calcein formed in the ladder microfluidic system, and theoretical 2-fold dilution concentration profile. FIG. 7E shows a comparison of the concentration profiles of resazurin, fluorescein, and Calcein loaded with their specific loading time and theoretical profile. Dash line with blue zone shows the theoretical concentration profile with 5% error. All data are shown as average±standard deviation.

FIGS. 8E-8C demonstrate the efficacy of the claimed microfluidic platform for AST of bacterial isolates from clinical veterinary samples. FIG. 8A shows a comparison of match vs. un-match between MICs obtained on-chip using the ladder microfluidic system and the gold standard method conducted at veterinary diagnostic lab. Targeted bacteria are Escherichia coli (EC), Proteus mirabilis (PRM), Enterococcus faecalis (EF), and Staphylococcus pseudintermedius (SP). FIG. 8B is table showing detailed percentage of matched vs. unmatched for each antibiotic/bacteria combination. FIG. 8C is a table showing the probability of obtaining an accurate MIC for each antibiotic/bacteria combination.

FIG. 9 is a Table demonstrating the efficacy of the ladder microfluidic platform for rapid AST of bacteria extracted directly from spiked and clinical canine urine samples. *EC Escherichia coli, PRM Proteus mirabilis, EF Enterococcus faecalis, SP Staphylococcus pseudintermedius, S spiked sample, C clinical sample, SVU Sensititre Veterinary UTI plate. Values highlighted in bold do not match.

FIGS. 10A-10D show detailed dimensions of embodiments of a ladder shaped microfluidic system (FIGS. 10A-10C) and length (L, in μm) of hydraulic constrictions on side channels (FIG. 10D).

FIG. 11 shows the fluorescent intensity patterns of resazurin in the microchambers at initial time and after 4 hours illustrating three possible scenarios: a minimum inhibitory concentration (MIC), antibiotic resistance, and invalid result due to no growth. Scale bar=100 μm.

FIG. 12 shows relative concentration profiles showing the kinetics of resazurin diffusion into a microchamber over time. Raw data used for calculation of the area under the curve of the concentration profile at each time point.

FIG. 13 is a table showing a linear correlation between the concentration profiles from computer simulation, of resazurin, fluorescein, and Calcein formed in an embodiment of a ladder microfluidic system, and theoretical 2-fold dilution concentration profile.

FIG. 14 is a table providing a summary of the antibiotics used in the Examples (infra) of the present disclosure. Source: NCI Thesaurus, DrugBank Online.

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure. In another example, reference to “a compound” includes both a single compound and a plurality of different compounds.

The term “about” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” depends on the particular system under study, and can be readily appreciated by one of ordinary skill in the art.

The term “and/or” as used herein means that the listed items are present, or used, individually or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

Certain terms employed in the specification, examples, and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

One aspect of the present disclosure relates to a microfluidic circuit comprising a drug inlet port; an outlet port; a drug inlet main channel fluidically connecting the drug inlet port and the outlet port, where said drug inlet main channel comprises (i) a plurality of serpentine mixers and (ii) a plurality of dead-end first microchamber sets; a negative inlet port; a negative inlet main channel fluidically connecting the negative inlet port and the drug inlet main channel; a plurality (n) of ladder channels, where each of the plurality (n) of the ladder channels is fluidically connected to both the drug inlet main channel and the negative inlet main channel; and an outlet channel fluidically connected to the drug inlet main channel between the drug inlet port and the outlet port.

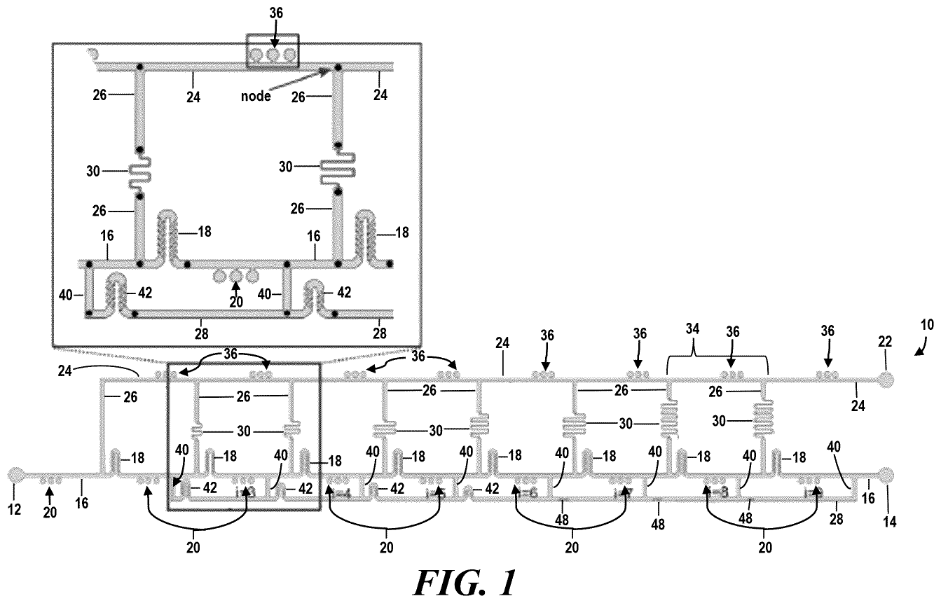

One embodiment of a microfluidic circuit of the present disclosure is illustrated in FIG. 1. FIG. 1 is a top view of one embodiment of a microfluidic circuit of the present disclosure. As illustrated in FIG. 1, microfluidic circuit 10 comprises drug inlet port 12, outlet port 14, and drug inlet main channel 16. Drug inlet main channel 16 fluidically connects drug inlet port 12 and outlet port 14.

Positioned in drug inlet main channel 16 are serpentine mixers 18 and dead-end first microchamber sets 20. In the embodiment illustrated in FIG. 1, eight serpentine mixers 18 are illustrated in drug inlet main channel 16, but any number of serpentine mixers may be used depending on the particular structure of the microfluidic circuit. The serpentine mixers may comprise jagged edges, as illustrated in FIG. 1 to create turbulence. In some embodiments, at least two serpentine mixers are positioned in the drug inlet main channel. In some embodiments, more than two serpentine mixers are positioned in the drug inlet main channel, e.g., 3, 4, 5, 6, 7, 8, 9, 10, or more serpentine mixers are positioned in the drug inlet main channel. In some embodiments, the number of serpentine mixers positioned in the drug inlet main channel depends on the degree of concentration gradient one wishes to achieve in the microfluidic surface.

With further reference to FIG. 1, microfluidic circuit 10 also includes negative inlet port 22 and negative inlet main channel 24. Negative inlet main channel 24 is fluidically connected to negative inlet port 22 and drug inlet main channel 16.

A plurality (n) of ladder channels, illustrated in FIG. 1 as ladder channels 26 are fluidically connected to both drug inlet main channel 16 and negative inlet main channel 24. In the particular embodiment illustrated in FIG. 1, drug inlet main channel 16 and negative inlet main channel 24 are positioned parallel to each other and fluidically connected by ladder channels 26 positioned between and perpendicular to inlet main channel 16 and negative inlet main channel 24.

Microfluidic circuit 10 of FIG. 1 also includes outlet channel 28, which is fluidically connected to drug inlet main channel 16 between drug inlet port 12 and outlet port 14.

In some embodiments, the microfluidic circuit of the present disclosure comprises a plurality of hydraulic resistor pinches. In the embodiment illustrated in FIG. 1, microfluidic circuit 10 comprises hydraulic resistor pinches 30 positioned in all but one of ladder channels 26. Specifically, ladder channel 26 located most proximate to drug inlet port 12 lacks a ladder channel, but all other ladder channels 26 comprise a hydraulic resistor pinch 30. In some embodiments, each of the ladder channels comprises a hydraulic resistor pinch. In some embodiments, such as the embodiment illustrated in FIG. 1, all but one of the ladder channels comprises a hydraulic resistor pinch. In some embodiments, more than one of the ladder channels lacks a hydraulic resistor pinch.

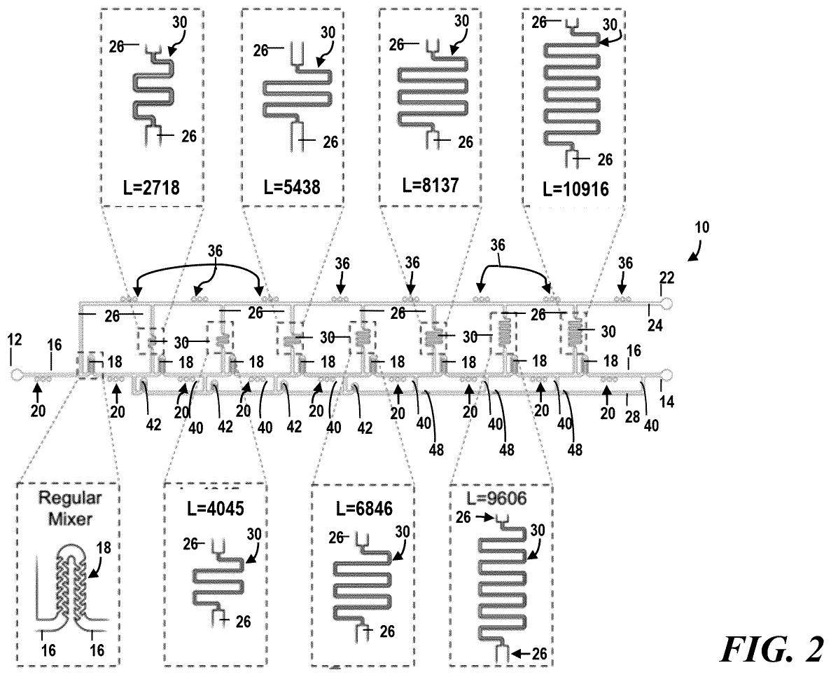

Examples of resistor pinches 30 that may be used in microfluidic circuit 10 of the present disclosure is illustrated in FIG. 2. FIG. 2 is also a top view of one embodiment of microfluidic circuit 10. The insets of FIG. 2 show greater details of resistor pinches 30 contained in microfluidic circuit 10. In particular, each of resistor pinches 30, positioned in separate ladder channels 26 are shown to have a different length, represented by “L.” As illustrated, the length of each resistor pinch 30 increases through microfluidic circuit 10 from left to right, or from drug inlet port 12 to outlet port 14, with the shortest resistor pinch 30 being most proximate to inlet port 12 and the longest resistor pinch 30 being most proximate to outlet port 14. As discussed in the Examples below, adjusting the length of the resistor pinches positioned in the ladder channels enables the creation of concentration gradients in dead-end first microchamber sets 20.

With reference again to FIG. 1, a subunit of microfluidic circuit 10 is shown in FIG. 1 as subunit 34. As illustrated, microfluidic circuit 10 comprises repeating subunits that form the circuit. A subunit, such as subunit 34, comprises a section of drug inlet main channel 16 and a section of negative inlet main channel 24 with two ladder channels 26. Each ladder channel 26 fluidically connects a section of drug inlet main channel 16 and a section of negative inlet main channel 24. In the embodiment illustrated in FIG. 1, subunit 34 also comprises a single serpentine mixer 18 positioned in negative inlet main channel 24 and a single hydraulic resistor pinch 30 in each of the two ladder channels 26.

The inset of FIG. 1 illustrates a portion of microfluidic circuit 10 in more detail. In particular, dead-end first microchamber sets 20 are illustrated. In the embodiment illustrated in FIG. 1, dead-end first microchamber set 20 comprises three distinct dead-end microchambers (illustrated as a circular chamber), each of which is fluidically connected to drug inlet main channel 16 via a side channel. Also, dead-end second microchamber sets 36 are illustrated. In the embodiment illustrated in the inset of FIG. 1, dead-end second microchamber set 36 comprises three distinct dead-end microchambers (illustrated as a circular chamber), each of which is fluidically connected to negative inlet main channel 24 via a side channel.

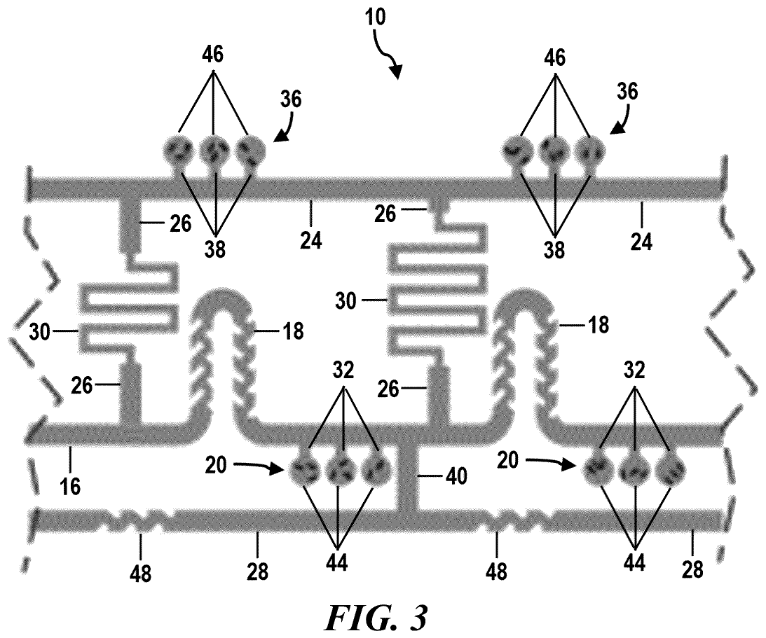

Each of dead-end first microchamber set 20 and dead-end second microchamber set 36 is illustrated in greater detail in FIG. 3. As illustrated in FIG. 3, dead-end first microchamber sets 20 each comprise three distinct microchambers 44 (comprising one set of dead-end first microchamber set 20). Each distinct microchamber 44 is fluidically connected to drug inlet main channel 16 via side channels 32. Similarly, dead-end second microchamber sets 36 each comprise three distinct microchambers 46 (comprising one set of dead-end second microchamber set 36). Each distinct microchamber 46 is fluidically connected to negative inlet main channel 24 via side channels 38.

In some embodiments of the microfluidic circuit according to the present disclosure, the drug inlet main channel 16 and the negative inlet main channel 24 are fixedly positioned parallel to each other.

In some embodiments of the microfluidic circuit according to the present disclosure, each of the ladder channels 26 is positioned perpendicular to each of the main channels (i.e., the drug inlet main channel 16 and the negative inlet main channel 24). In some embodiments, the ladder channels 26 may be fixedly positioned parallel to each other between the drug inlet main channel and the negative inlet main channel.

In some embodiments of the microfluidic circuit according to the present disclosure, n−1 of the plurality (n) of ladder channels 26 each comprises a hydraulic resistor pinch 30. In some embodiments, the ladder channel positioned most proximate to the drug inlet port does not possess a hydraulic resistor pinch 30. In some embodiments, the hydraulic resistor pinch 30 in each of the ladder channels 26 is of a different length than the other ladder channels 26.

In some embodiments where the hydraulic resistor pinch 30 in each of the ladder channels 26 is of a different length than the other ladder channels 26, the length of each hydraulic resistor pinch 30 increases in the direction of the drug inlet port 12 to the outlet port 14. In some embodiments, the length of each hydraulic resistor pinch 30 is between 200 μm-10,000 μm or 2000 μm-10,000 μm.

In some embodiments of the microfluidic circuit according to the present disclosure, each of the microchambers 44 in the plurality of dead-end first microchamber sets 20 is individually fluidically connected to the drug inlet main channel 16 by a side channel 32.

The plurality of dead-end first microchamber sets 20 may comprise any suitable shape or size. In the embodiments illustrated in the drawings referenced herein, the microchamber sets have a circular shape, although other shapes may also be contemplated. In some embodiments, microchambers 44 are circular in shape. In some embodiments the dead-end first microchamber sets 20 comprise microchambers 44 that have a volume capacity of about 50 μm, about 100 μm, about 200 μm, about 300 μm, about 400 μm, or about 500 μm. In some embodiments, side channels 32 have a length between the main channel they are connected to and the microchamber of about 20 μm, about 30 μm, about 40 μm, about 50 μm, about 60 μm, about 70 μm, about 80 μm, about 90 μm, about 100 μm, about 110 μm, or about 120 μm.

In some embodiments of the microfluidic circuit according to the present disclosure, a pair of ladder channels 26 in sequence between the drug inlet main channel 16 and the negative inlet main channel 24 comprises a subunit 34 of the microfluidic circuit 10. In accordance with such embodiments, each subunit may comprise only one of the dead-end first microchamber sets 20. In some embodiments, at least one of the subunits 34 comprises a plurality of dead-end second microchamber sets 36. Each of the dead-end second microchambers 46 of the dead-end second microchamber sets 36 may be individually fluidically connected to the negative channel by a side channel 38. In some embodiments, the plurality of dead-end second microchamber sets 36 is comprised of three distinct microchambers 46.

In some embodiments of the microfluidic circuit according to the present disclosure, the plurality of dead-end first microchamber sets 20 is comprised of three distinct microchambers 44.

In some embodiments of the microfluidic circuit according to the present disclosure, the outlet channel 28 is positioned parallel to the drug inlet main channel 16. The outlet channel 28 may be fluidically connected to the drug inlet main channel 16 by one or more outlet side channels 40. In some embodiments, only a single outlet side channel 40 is positioned in the drug inlet main channel 16 between proximate pairs of the ladder channels.

In some embodiments of the microfluidic circuit according to the present disclosure, the outlet channel 28 comprises a plurality of serpentine mixers 42. In some embodiments, only a single serpentine mixer 42 is positioned in the outlet channel 28 between proximate pairs of the ladder channels 26.

The microfluidic circuit of the present disclosure pertains to a fluidic system of ports, channels, and microchambers, all fluidically connected. Since, as discussed in the Examples below, the particular structure of this fluidically connected system enables assays to be performed based on the unique structure of the circuit, the circuit may be used in a variety of contexts or on a variety of platforms. One particular platform where the microfluidic circuit of the present disclosure is useful is a chip-like platform. For example, the microfluidic circuit may be formed into a planar material to create the system of ports, channels, and microchambers, accessible for input or output of fluid only through the ports.

Thus, another aspect of the present disclosure relates to a microfluidic device comprising a support layer; a substrate layer disposed on the support layer; and a microfluidic circuit of the present disclosure, where the microfluidic circuit is disposed within the substrate layer.

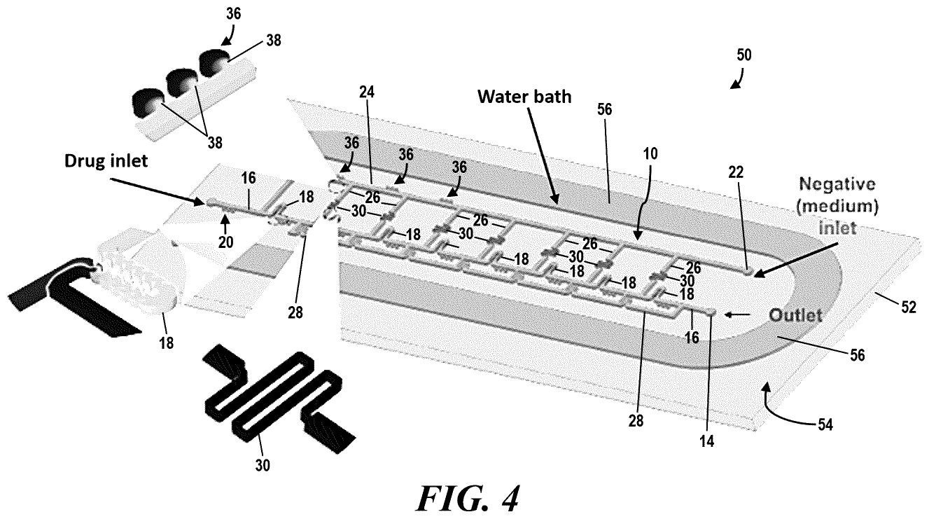

With reference now to FIG. 4, illustrated is a perspective view of one embodiment of a microfluidic device of the present disclosure. Specifically, microfluidic device 50 is illustrated. Microfluidic device 50 includes support layer 52, substrate layer 54, and microfluidic circuit 10 disposed on support later 52. In some embodiments, microfluidic device 50 comprises water bath 56 disposed within substrate layer 54, where water bath 56 is separate from and proximate to microfluidic circuit 10. Water bath 56 may comprise porous walls to allow water vapor exchange.

In some embodiments of microfluidic device 50 according to the present disclosure, drug inlet main channel 16 and negative inlet main channel 24 may be fixedly positioned parallel to each other.

In some embodiments of the microfluidic device according to the present disclosure, each of ladder channels 26 may be positioned perpendicular to each of the main channels (i.e., drug inlet main channel 16 and negative inlet main channel 24). For example, ladder channels 26 may be fixedly positioned parallel to each other between the drug inlet main channel and the negative inlet main channel.

In some embodiments of microfluidic device 50 according to the present disclosure, n−1 of the plurality (n) of ladder channels 26 each comprises a hydraulic resistor pinch 30. In accordance with such embodiments, ladder channel 26 positioned most proximate to the drug inlet port may not possess a hydraulic resistor pinch 30 and/or hydraulic resistor pinch 30 in each of ladder channels 26 may be of a different length than the other ladder channels. In some embodiments, the length of each hydraulic resistor pinch 30 increases in the direction of drug inlet port 12 to outlet port 14.

In some embodiments of microfluidic device 50 according to the present disclosure, each of the microchambers in the plurality of dead-end first microchamber sets is individually fluidically connected to the drug inlet main channel by a side channel.

In some embodiments of microfluidic device 50 according to the present disclosure, a pair of ladder channels 26 in sequence between drug inlet main channel 16 and negative inlet main channel 24 comprises a subunit of microfluidic circuit 10. In some embodiments, each subunit 34 comprises only one of dead-end first microchamber sets 20. In other embodiments, at least one of subunits 34 comprises a plurality of dead-end second microchamber sets 36. In some embodiments, each of dead-end second microchambers 46 of dead-end second microchamber sets 36 is individually fluidically connected to negative inlet main 24 channel by side channel 38. In some embodiments, plurality of dead-end second microchamber sets 36 is comprised of three distinct microchambers 46.

In some embodiments of microfluidic device 50 according to the present disclosure, plurality of dead-end first microchamber sets 20 is comprised of three distinct microchambers 44.

In some embodiments of microfluidic device 50 according to the present disclosure, outlet channel 28 is positioned parallel to drug inlet main channel 16.

Outlet channel 28 may be fluidically connected to drug inlet main channel 16 by one or more outlet side channels 40. In some embodiments, only a single outlet side channel 40 is positioned in drug inlet main channel 16 between proximate pairs of the ladder channels.

In some embodiments of microfluidic device 50 according to the present disclosure, outlet channel 28 comprises a plurality of serpentine mixers 42. In some embodiments, only a single serpentine mixer 42 is positioned in outlet channel 28 between proximate pairs of ladder channels 26.

A variety of materials may be used to form the solid support and/or a substrate, which itself is formed on or connected to the solid support. The support and/or substrate materials should be selected based upon their compatibility with known microfabrication techniques, for example, photolithography, 3-D printing, wet chemical etching, laser ablation, air abrasion techniques, injection molding, embossing, and other techniques, or based on the application being used. The support and/or substrate materials are also generally selected for their compatibility with the full range of conditions to which the microfluidic devices may be exposed, including extremes of pH, temperature, salt concentration, and/or application of electric fields, should these be relevant in performing assays using the microfluidic device of the present disclosure.

In some embodiments, suitable support and/or substrate materials include, without limitation, glass, pyrex, glass ceramic, polymer materials, semiconductor materials, and combinations thereof. In some embodiments, the support and/or substrate material may include materials normally associated with the semiconductor industry in which microfabrication techniques are regularly employed, including, e.g., silica based substrates such as glass, quartz, silicon, or polysilicon, as well as other substrate materials, such as gallium arsenide and the like. In some embodiments, the support layer comprises glass.

Exemplary polymeric materials include, without limitation, plastics such as polymethylmethacrylate (PMMA), polycarbonate, polytetrafluoroethylene (TEFLON™), polyvinylchloride (PVC), polydimethylsiloxane (PDMS), and polysulfone. Other plastics can also be used. Such materials are readily manufactured from microfabricated masters, using well known molding techniques, such as injection molding, embossing or stamping, or by polymerizing the polymeric precursor material within a mold. Such polymeric substrate materials are known for their case of manufacture, low cost and disposability, as well as their general inertness to most extreme reaction conditions. These polymeric materials may include treated surfaces, for example, derivatized or coated surfaces, to enhance their utility in the microfluidic device or, for example, to provide enhanced fluid direction should that be a needed factor in carrying out assays using microfluidic devices described herein. In some embodiments, the substrate layer comprises PDMS.

As described in more detail supra, there is a need to develop new platforms for rapid, inexpensive, and easily implemented antibiotic resistance testing. Accordingly, a further aspect of the present disclosure relates to a method for performing an assay. This method involves loading a test solution into a microfluidic device of the present disclosure. A culture medium containing an antibiotic solution is loaded into the drug inlet port, a culture medium is loaded into the negative medium inlet port, and the device is then washed with oil to isolate each of the plurality of microchambers. The method further involves detecting a fluorescent signal in each of the plurality of microchambers.

In some embodiments, the test solution comprises a metabolic dye. Suitable metabolic dyes include, without limitation, resazurin.

In some embodiments, the test solution comprises a biological sample. The biological sample may be in solution, suspension, or solid form. In some embodiments, the test solution comprises a bacterial suspension.

Suitable biological samples include, without limitation, blood, urine, saliva, sputum, and cerebral spinal fluid.

The test solution and/or biological sample may comprise one or more bacterial species. In some embodiments, the test solution and/or biological sample comprises a single bacterial species.

The bacterial species may be a pathogenic bacterial species. Suitable exemplary pathogenic bacterium include, without limitation, Escherichia coli, Proteus mirabilis, Enterococcus faecalis, Staphylococcus pseudintermedius, Salmonella, Staphylococcus aureus, Streptococcus pyogenes, Mycobacterium tuberculosis, Clostridium botulinum, Clostridium difficile, Neisseria meningitidis, Neisseria gonorrhoeae, Helicobacter pylori, Vibrio cholerae, Bacillus anthracis, Yersinia pestis, Listeria monocytogenes, Legionella pneumophila, Shigella, Campylobacter jejuni, Haemophilus influenzae, Bordetella pertussis, Brucella, Francisella tularensis, Rickettsia rickettsii, Chlamydia trachomatis, Treponema pallidum, Borrelia burgdorferi, Vibrio vulnificus, Acinetobacter baumannii, Pseudomonas aeruginosa, and Klebsiella pneumoniae.

In some embodiments, the test solution and/or biological sample contains an unknown bacterial species.

In some embodiments, the test solution comprises at least 10⁴ CFU/mL, 10⁵ CFU/mL, or 10⁶ CFU/mL. For example, the test solution may comprise at least 10⁵ CFU/mL of a bacterial species.

Loading the test solution into the microfluidic device may be carried out by loading the test solution through the drug inlet port and/or the negative inlet port. In some embodiments, loading the test solution into the microfluidic device is carried out through the drug inlet port while the remaining openings (i.e., the negative inlet port and the outlet port) are blocked. Alternatively, loading the test solution into the microfluidic device is carried out through the negative inlet port while the remaining openings (i.e., the drug inlet port and the outlet port) are blocked; or through the outlet port while the remaining openings (i.e., the negative inlet port and the drug inlet port) are blocked. Loading the test solution is carried out to completely fill the device and load the microchambers.

Loading the culture medium comprising the antibiotic solution is carried out through the drug inlet port. In some embodiments, the culture medium comprising the antibiotic solution is carried out at a flow rate of 1-1000 μL/hour, or any amount therebetween. In some embodiments, the culture medium comprising the antibiotic solution is carried out at, e.g., 35 μL/hour.

Loading the culture medium is carried out through the negative medium inlet port at a flow rate of 1-1000 μL/hour, or any amount therebetween. In some embodiments, the culture medium comprising the antibiotic solution is carried out at, e.g., 262 μL/hour.

The culture medium comprising the antibiotic solution and the culture medium may be loaded simultaneously or sequentially. In some embodiments, the culture medium comprising the antibiotic solution and the culture medium are loaded simultaneously.

Loading the culture medium comprising the antibiotic solution and the culture medium may be carried out for 1-10 minutes or more. In some embodiments, the culture medium comprising the antibiotic solution and the culture medium are loaded for about 1 minute, about 2 minutes, about 3 minutes, about 4 minutes, about 5 minutes, about 6 minutes, about 7 minutes, about 8 minutes, or about 10 minutes.

Loading the culture medium comprising the antibiotic solution through the drug inlet port and loading the culture medium through the negative inlet port as described in the present disclosure enables the generation of serial 2-fold concentration gradients in the dead-end first microchamber sets of the microfluidic device of the present disclosure.

Suitable antibiotics for use in the method according to the present disclosure include, without limitation, penicillin (e.g., amoxicillin, ampicillin, and penicillin V), cephalosporin (e.g., cefalexin, cefuroxime, ceftriaxone, ceftiofur), macrolides (e.g., azithromycin, clarithromycin, and crythromycin), fluoroquinolones (e.g., ciprofloxacin, enrofloxacin, levofloxacin, and moxifloxacin), tetracyclines (e.g., doxycycline and tetracycline), aminoglycosides (e.g., gentamicin and tobramycin), and sulfonamides (e.g., sulfamethoxazole/trimethoprim). In some embodiments, the antibiotic solution comprises a single antibiotic. In other embodiments, the antibiotic solution comprises a combination of at least two antibiotics, at least three antibiotics, or at least four antibiotics. For example, the antibiotic solution may comprise Amoxicillin and Clavulanic acid or Trimethoprim and Sulfamethoxazole.

Washing the device with oil may be carried out through the drug inlet port and/or negative inlet port.

In some embodiments of the methods of the present disclosure, the method further involves incubating the microfluidic device. The microfluidic device may be incubated at, e.g., 37° C. Said incubating may be carried out for about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more. In some embodiments, the microfluidic device is incubated for about 4 hours to about 5 hours.

In some embodiments, detecting a fluorescent signal in each of the plurality of microchambers is carried out at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more after said washing step. For example, a fluorescent signal in each of the plurality of microchambers may be carried out after about 4 to 5 hours following the washing step.

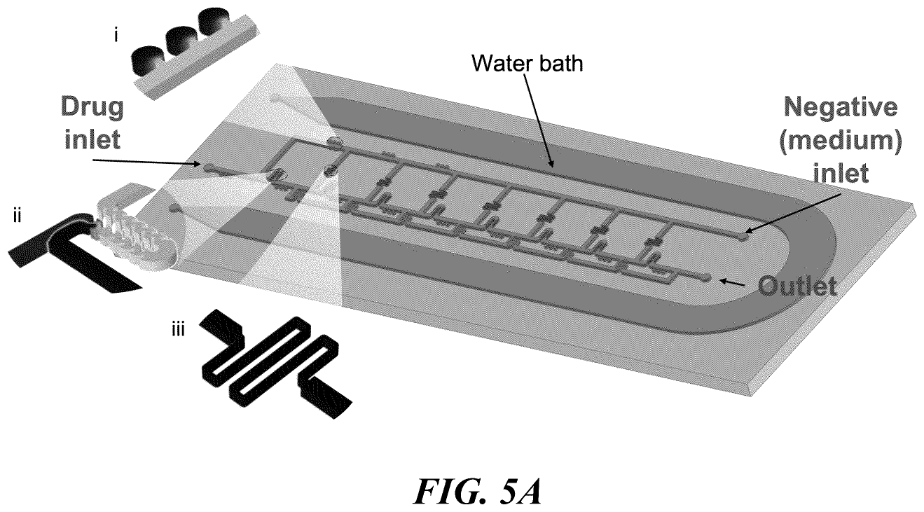

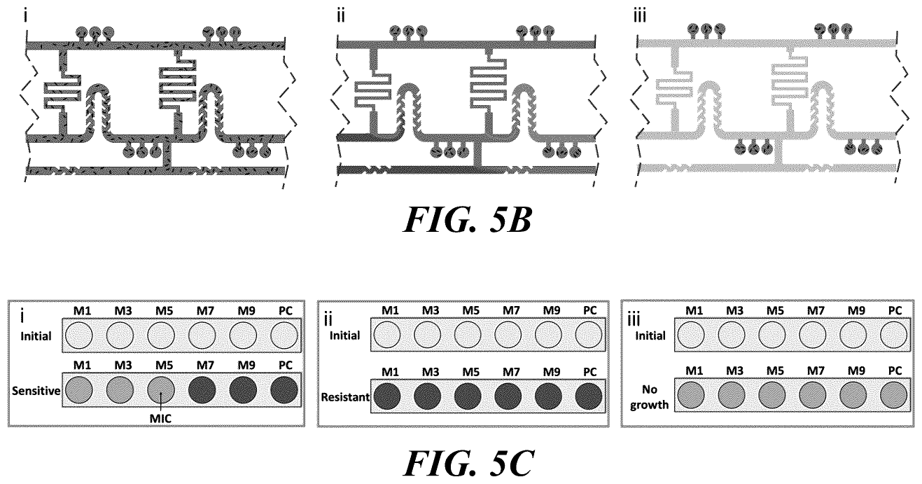

In operation, the test solution is loaded into the microfluidic device using a syringe. With reference to FIG. 1 and FIG. 5B (left panel), loading the test solution completely fills each of dead-end first microchamber sets 20 and each of dead-end second microchamber sets 36 with the test solution. The culture medium comprising the antibiotic solution and the culture medium are loaded through drug inlet port 12 and negative inlet main channel 22, respectively. Loading of the culture medium comprising the antibiotic solution and the culture medium is carried out using syringe pumps to generate a concentration gradient in the microfluidic device, with dead-end first microchamber set 20 closest to drug inlet port 12 comprising the highest concentration of the antibiotic solution and dead-end first microchamber set 20 closest to the negative inlet port comprising the lowest concentration of the antibiotic solution. Dead-end second microchamber sets 36 serve as negative controls. With reference to FIG. 3 and FIG. 5B (middle panel), loading of the culture medium comprising the antibiotic solution and the culture medium washes away the bacteria in drug inlet main channel 16, serpentine mixers 18 and 42, negative inlet main channel 24, ladder channels 26, hydraulic resistor pinches 30, side channels 32, 38, and 40, but retains bacteria inside dead-end microchambers 20 and 36. Antibiotic next diffuses into each of dead-end first microchamber sets 20 according to a drug specific loading time. With reference to FIG. 5B (right panel), the microfluidic device is washed with oil (e.g., biological grade mineral oil) to isolate each of dead-end first microchamber sets 20 and dead-end second microchamber sets 36. With reference to FIG. 4, water bath 56 is loaded with water and the microfluidic device is loaded with water, followed by incubation at 37° C. to allow detection of a minimum inhibitory concentration of the antibiotic solution.

In some embodiments, the method further involves calculating a minimum inhibitory concentration based on said detecting.

### EXAMPLES

### Materials and Methods for Examples 1-5

### Clinical Samples

Bacterial isolates (30) from canine urinary tract infections were obtained from the Cornell Animal Health Diagnostic Center (AHDC). The isolates were identified to species level, with antibiotic susceptibilities examined using a Sensititre™ system (SVU) with a urinary plate for isolates of veterinary origins. The 30 isolates include Escherichia coli (n=14), Proteus mirabilis (n=8), Staphylococcus pseudintermedius (n=5), Enterococcus faecalis (n=3), and were selected to reflect prevalence of these organisms as observed at Cornell AHDC in canine urine. Isolates were subcultured onto fresh blood agar plate prior to testing and kept at refrigeration temperature for no longer than 5 days.

For direct-from-sample testing, de-identified discarded canine urine from AHDC was obtained. Samples were refrigerated overnight until confirmed for the presence of bacterial growth by the diagnostic center. Among the samples, four were growth negative and seven were growth positive. The inclusion criteria for growth positive samples were single organism growth with a count of at least 10⁵ CFU mL⁻¹. With the exception of swabs (as this method does not yield fluid samples), no sample collection methods were excluded. The antibiotic susceptibilities of these pathogens bacteria were examined by the AHDC using the Sensititre™ system (SVU) and the microfluidic system according to the present disclosure, and the results were compared.

### Antibiotics

Seven different antibiotics and antibiotic combinations were tested, following those included in the SVU plate. They were Enrofloxacin, Ceftiofur, Tetracycline, Cefalexin, Ampicillin, and two antibiotic combinations Amoxicillin/Clavulanic acid 2:1 ratio, and Trimethoprim/Sulfamethoxazole 8:152 ratio. Antibiotic solutions were prepared from powdered stocks then aliquoted and stored at −20° C.

### Microfluidic Device Fabrication

The microfluidic device was made from polydimethylsiloxane (PDMS) (Silgard 184; Dow Corning) patterned with standard soft lithography technique and permanently bonded with a glass substrate (Xia & Whitesides, “Soft Lithography,” Angew. Chem. Int. Ed. 37:550-575 (1998), which is hereby incorporated by reference in its entirety). SU-8 2075 was used to make the silicon mold. PDMS was mixed at 1:10 ratio of curing agent to polymer and cured at 70° C. for 2 hours. The PDMS and glass substrate was treated with plasma for 1 minute to make a permanent bond, and then the device was incubated at 70° C. for at least another 30 minutes to promote the bonding.

### Characterization of the Device

Resazurin was used to characterize the diffusion of molecules into the microchambers. Fluorescent images were taken using a fluorescent microscope (ZOE™ Fluorescent Cell Imager system; Bio-rad, Hercules, California) every 30 seconds up to 5 minutes, then analyzed using ImageJ. To measure the concentration gradient experimentally, resazurin solutions were loaded for a resazurin-specific loading time, then the main channel was washed with mineral oil to isolate the microchambers. The fluorescent intensities of the resazurin in the microchambers were analyzed using ImageJ software. Because the concentration profile on the device expands across more than two orders of magnitude (100%-0.3%), three resazurin solutions (i.e., 10 μg mL⁻¹, 1 μg mL⁻¹, 0.1 μg mL⁻¹) were used to characterize the concentration gradient in three zones: high (M1-3), medium (M3-6), and low (M6-9). At each microchamber, the relative fluorescent signal compared to the previous microchamber was determined, then the concentration at each was calculated. To examine the ability to generate the designed concentration gradient for different molecules, fluorescein and Calcein were loaded using their molecule-specific loading time, then calculated the concentration profiles generated.

### Numerical Simulation and Network Model Solver

Comsol multiphysics 5.4a software was used for Finite Element Analysis of the equivalent constriction length to the hydraulic resistance of the mixer. For the detailed characterization of the network, a generalized computational method was developed to calculate the flow rates and concentrations. The algorithm resembled the Hardy Cross method of momentum distribution through pipe networks (Cross, H., “Analysis of Flow in Networks of Conduits or Conductors,” https://www.ideals.ilinois.edu/items/4876 (1936), which is hereby incorporated by reference in its entirety). It was unrealistic to hand-calculate the flow rates and concentrations for all 65 nodes on the chip.

All nodes, including both ends of the features in the chip and the channel junctions, are assigned a unique number between one and the total number of the nodes (n). An Excel sheet should be generated with the first column starting node (i) and second column ending node (j) for each feature. Each row contains width and length of the feature and in the starting node (i) and ending node (j) the numbers should be placed in increasing order. The subroutine then reads the excel sheet and creates a resistor matrix (R) in which the row index would be the number of the starting node and the column index is the ending node and the value is the resistance based on the following formula.

$$R = \frac{12 L}{{HW}^{3}} \qquad ( 4 )$$

L, W and H are the length, width, and height of the feature respectively.

A second sheet in the Excel file was named “PQC” that contains the boundary conditions of pressure, flow rate and concentration (Table 1).

TABLE 1
Excel Spreadsheet Layout for Determination
of Pressure, Flow Rate, and Concentration

| | Boundary Conditions | | |
|---|---|---|---|
| Node type | Pressure | Flow Rate | Concentration |
| Inlet nodes | Leave empty | + if incoming | Should be known value |
| | | − if outgoing | |
| Outlet nodes | 0 | Leave empty | Leave empty |
| Internal nodes | Leave empty | Leave empty | Leave empty |

Using these two sheets, a matrix will be created containing C(n,2)+n rows and columns, in which C(n,2) is the binomial coefficient of 2 from n for each combination of (i,j) from 1 to n. This is the coefficient matrix for the flow rates between each two nodes plus the pressure at each node which are solved for. The first C(n,2) elements are related to flow rates between each two nodes (zero if there is no connection between the nodes) and the remaining n would be the pressures. The following equation regarding the balance of pressure for the connection between i and j can be written:

$${P}_{i} - {R}_{i , j} {q}_{i , j} = {P}_{j} \qquad ( 5 )$$

for all the combinations of i and j or if nothing is known then

Pᵢ−Pⱼ−R_{i,j}q_{i,j}=0.

If there is an external flow source attached to a node, it can be assumed that there is a ghost node outside of the network that is connected to the node via a resistor with resistance of 1. Therefore, the right-hand side of the above equation becomes

$${P}_{i} - {P}_{j} - {R}_{i , j} {q}_{i , j} = - 1 \times {q}_{e} \qquad ( 6 )$$

in which qₑ is the external flow source amount. Another set of n equations is the mass balance at each node.

Σₖ₌₁ⁿqₖ=0 because all incoming flows rates to a node are positive and the outgoing flow rates are negative such that at each node the flow rate sums to zero. With these C(n,2)+n equations the set would be closed and can be solved for pressures and flow rates. Once these are calculated, assuming that all the incoming flows mix thoroughly, concentrations at each node can be solved for.

$${C}_{k} = \frac{{\sum }_{j}^{q > 0} {C}_{j} \times {q}_{j , k}}{{\sum }_{j}^{q > 0} \times {q}_{j , k}}$$

In which Σⱼ^{q>0}q_{j,k} means summation over all the neighbor nodes that have incoming flow rates into the node; that is q_{j,k}>0. To understand how the indexes are managed refer to the code itself.

### On-Chip AST

The device loading procedure is similar to protocols in previous studies (Nguyen et al., “Diffusion-Convection Hybrid Microfluidic Platform for Rapid Antibiotic Susceptibility Testing,” Anal. Chem. 93:5789-5796 (2021); Azizi et al., “Biological Small-Molecule Assays Using Gradient-Based Microfluidics,” Biosens. Bioelectron. 178:113038 (2021); and Azizi et al., “Antimicrobial Susceptibility Testing in a Rapid Single Test via an Egg-Like Multivolume Microchamber-Based Microfluidic Platform,” ACS Appl. Mater. Interfaces 13:19581-19592 (2021), which are hereby incorporated by reference in their entirety). In brief, bacterial inoculum was loaded into an empty device from one of the openings (e.g., the C₀ inlet), while the remaining openings were blocked, to remove air bubbles and to completely fill the device specifically in the microchambers. Then, C₀ solution containing a maximum concentration of an antibiotic, and culture medium alone were loaded into the device from two different inlets at flow rates 35 μL h⁻¹ and 262 μL h⁻¹, respectively (flow rate ratio of ˜7.5). The two solutions were loaded for a specific time depending on the antibiotic, to make the designed concentration gradient which diffuses into the microchambers. For most of the antibiotics tested, the loading time was under 6 minutes, however, the loading time for the Amoxicillin/Clavulanic acid and the Trimethoprim/Sulfamethoxazole antibiotic combinations were set at 10 minutes. A longer loading time for antibiotic combinations ensures that both antibiotics have saturated the microchambers, as each has a different diffusion rate. Finally mineral oil was loaded from the inlet to wash out the solutions, create an oil/water cap on the microchambers, and prevent contamination. Prior to incubation, water was added to the surrounding water bath through one of its openings, which helps to reduce evaporation of the reaction inside the microchambers by water vapor exchanging through the porous PDMS walls. All AST testing using the presently disclosed microfluidic system follows the following procedures. First, the bacterial suspension was prepared and calibrated to a McFarland 0.5 standard using a spectrophotometer, which correlates to a concentration of 1.5×10⁸ CFU mL⁻¹. For the study using bacterial isolates, the suspension was prepared by resuspending 2-3 colonies in sterile PBS. Then, 30 μL of the bacterial suspension was added to 1 mL of culture medium (final inoculum concentration ˜3×10⁶ CFU mL⁻¹). In all AST experiments, Mueller-Hinton broth supplemented with 2 v/v % PrestoBlue Cell Viability Reagent was used as a culture medium. Each C₀ antibiotic solution was prepared by diluting frozen stocks in culture medium to a concentration 2 times that of the maximum concentration on the SVU plate, except for Amoxicillin/Clavulanic acid and Trimethoprim/Sulfamethoxazole antibiotic combinations were diluted to the same as the maximum concentrations on the plate. One device was used for each antibiotic/bacteria combination, nine 2-fold diluted antibiotic concentrations were examined, and one positive control where bacteria were not exposed to the antibiotic was included. The bacteria was loaded, the antibiotic concentration gradient was generated, and the oil was loaded as described supra. Extra oil was added to the top of the inlet/outlet to make an oil droplet and help prevent air from entering the system and disrupting the oil/culture medium interface. The devices were placed into a petri dish with enough water to cover the glass substrate and incubated at 37° C. in an incubator. After 4 hours, the red fluorescent intensities of the microchambers on each device were examined to determine the MIC using a fluorescent microscope (ZOE™ Fluorescent Cell Imager system; Bio-rad, Hercules, California). For slow growth samples where the MIC was not clear after 4 hours incubation, samples were incubated for an additional 30 minutes to 1 hour.

### AST on Spiked and Clinical Urine Samples

Spiked urine samples were prepared by spiking overnight MHB-culture of bacteria into pooled negative growth urines at 10⁶ CFU mL⁻¹. The spiked samples were refrigerated for approximately the same duration that the clinical urine samples would be kept prior to AST testing in our laboratory. Seven of the 30 bacterial isolates were randomly chosen for the spiking study. The bacteria from both spiked and positive growth urine samples were retrieved by a two-step centrifugation/filtration method. First, 2 mL samples were centrifuged at 13,000 g for 1 minute, and the pellet was resuspended in 1 mL fresh MHB. Then, the solution was filtered through a 5 μm filter to remove debris and non-bacterial cells. Afterward, the bacterial solution was incubated at 37° C. with shaking at 120 rpm for 2 hours. Subsequent inoculum preparation and AST were performed as described.

### Statistics and Reproducibility

JMP version 16 was used to fit a generalized linear model to assess the probability of the two methods matching and how that depended on the drug and the bacteria tested. A binomial distribution with a logit link was used, and effects of antibiotic, bacteria, and their interaction were fixed. Because certain drug-bacteria combinations had a 100% match rate, Firth Bias-Adjusted Estimates was used. Other experiments on fluorescent dyes kinetic and concentration gradients were conducted with independently prepared replicates. Data are presented as means and standard deviations.

### Example 1—Description of the System

The ladder microfluidic system for antibiotic susceptibility testing (AST) combines a method of nanoliter microchamber-based AST (Nguyen et al., “Diffusion-Convection Hybrid Microfluidic Platform for Rapid Antibiotic Susceptibility Testing,” Anal. Chem. 93:5789-5796 (2021) and Azizi et al., “Nanoliter-Sized Microchamber/Microarray Microfluidic Platform for Antibiotic Susceptibility Testing,” Anal. Chem. 90:14137-14144 (2018), which are hereby incorporated by reference in their entirety) with a ladder-shape concentration gradient generator (LCGG). This combination provides a standardized and tunable antibiotic concentration profile for rapid phenotypic AST (FIG. 5A). The platform consists of a patterned polydimethylsiloxane (PDMS) layer bonded to a conventional glass slide, allowing testing of one antibiotic/bacteria combination per device. Each device provides ten 2-fold diluted concentrations for testing in triplicate microchambers for each concentration including positive growth control (no antibiotic) microchambers (FIG. 5A, (i)). The microchambers branch from the main channels of the LCGG and serve as bioreactors to incubate bacteria with antibiotics.

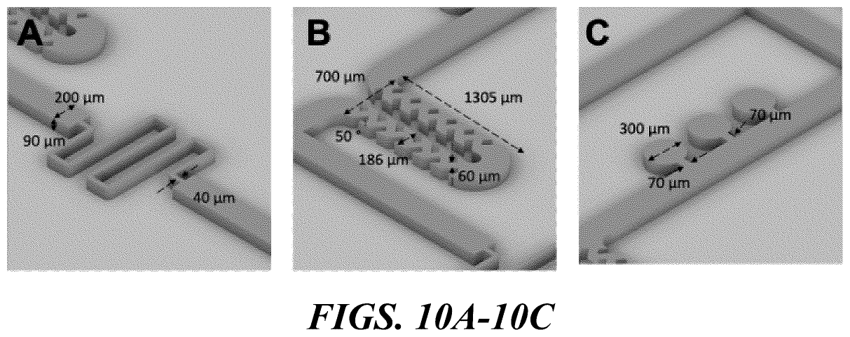

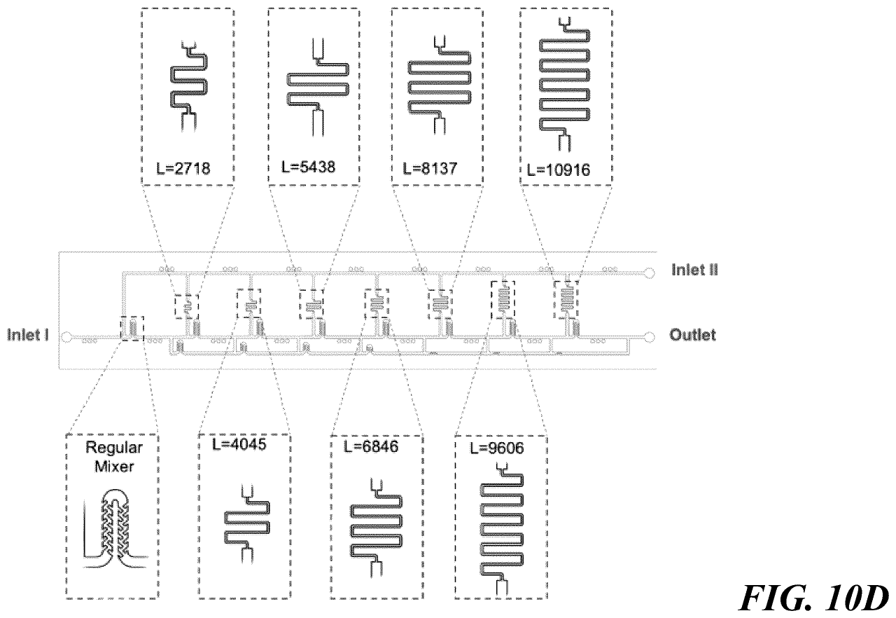

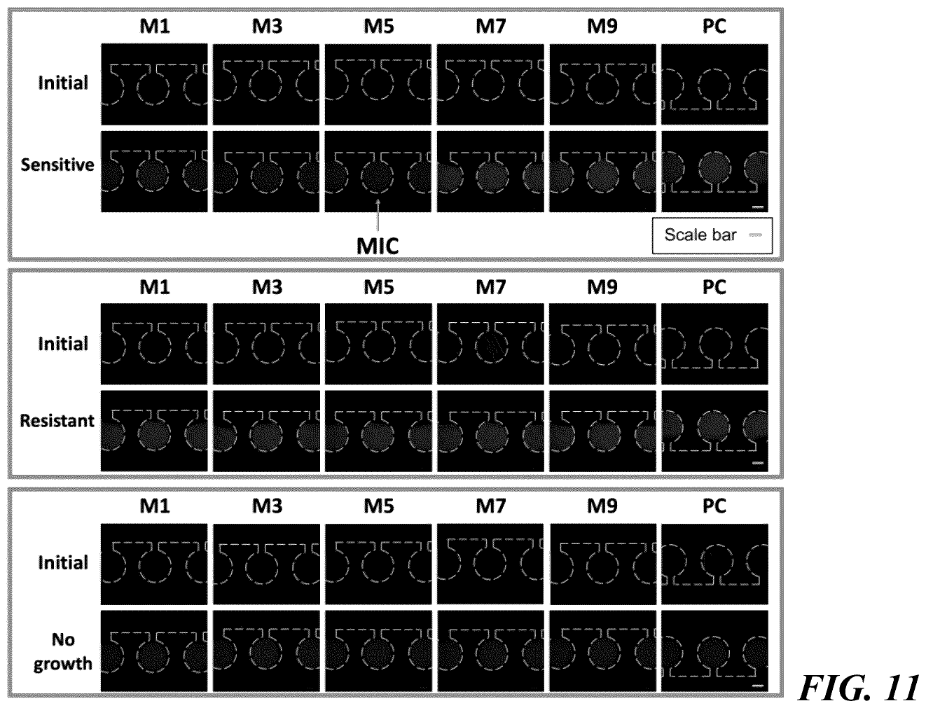

The device has three openings: a drug inlet (culture medium containing antibiotic), a negative inlet (pure culture medium), and an outlet (detailed dimensions in FIGS. 10A-10D). The drug inlet main channel and the negative inlet main channel are connected by side channels, creating a ladder-like structure, the LCGG. The LCGG controls the convection of fluid to generate a target concentration profile without using external valves; instead connecting loops are used where each loop results in one 2-fold dilution. The antibiotic and culture medium are added to the device from opposite directions, and portions of the negative solution flow to the drug side through the side channels and dilute the incoming drug solution at each node. The antibiotic is sequentially diluted as it moves through the system (from drug inlet to negative inlet). To ensure that the diluting antibiotic solution is homogeneous before exposing it to the bacteria, a modified serpentine is used (FIG. 5A, (ii)), to mix the drug and culture media in a specific range of flow rates. An on-chip water bath feature surrounds the entire main device, allowing water vapor exchange through the porous PDMS wall and reducing reagent evaporation during incubation.

### Example 2—Design and Simulation of the Device

A critical engineering challenge of the device was matching the flow rate of the diluter (pure culture media) to the incoming antibiotic stream and to create the 2-fold dilution at each loop. Without any control feature, the diluter would reach only half of the loops as the path of least resistance is a close-by outlet where the fluid can flow out of the system. This was solved by increasing the hydraulic resistances on the side channels, thus driving the diluter to reach all the loops. The approximate value of the resistance which results in desired concentration profile in the designed ladder layout can be calculated using circuit logic modeling Nguyen et al., “Diffusion-Convection Hybrid Microfluidic Platform for Rapid Antibiotic Susceptibility Testing,” Anal. Chem. 93:5789-5796 (2021) and Oh et al., “Design of Pressure-Driven Microfluidic Networks Using Electric Circuit Analogy,” Lab. Chip 12:515-545 (2012), which are hereby incorporated by reference in their entirety). Initially the resistance of the main channels was ignored due to their larger dimensions with respect to the rest of the chip features. A more detailed model was later used to evaluate the performance of the design, which included the resistance of the main channels.

At the side channels the flow rates were designed to be a half that of the drug inlet flow rate. For simplicity flow rates are referred to as their ratio to the drug inlet. At each loop, a relative flow rate, 0.5 of the drug, is mixed with a flow rate of 0.5 of the diluent coming from the top main channel. After this mixture passes through the mixer it flows into a series of 3 microchambers which are attached to the main channel. Right after these microchambers, 0.5 of this flow rate will be removed through a small added section.

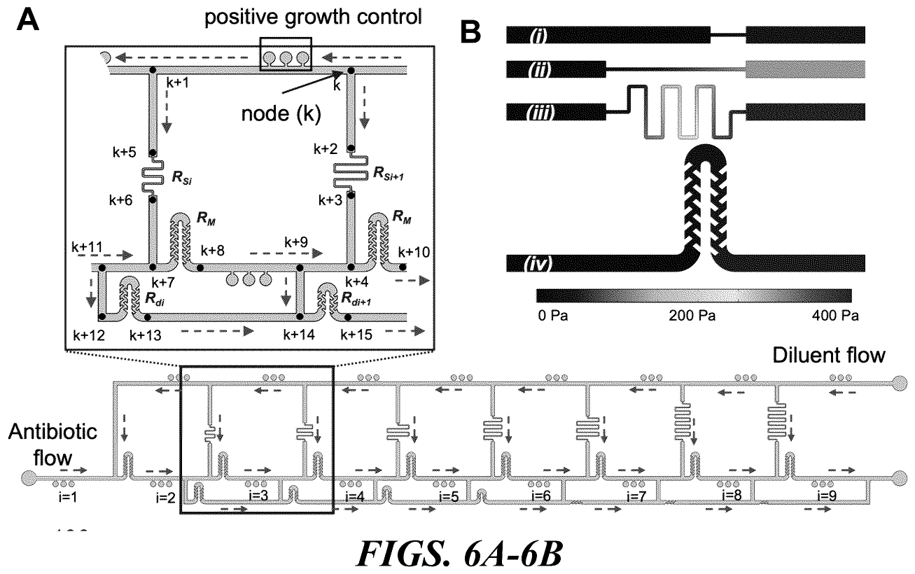

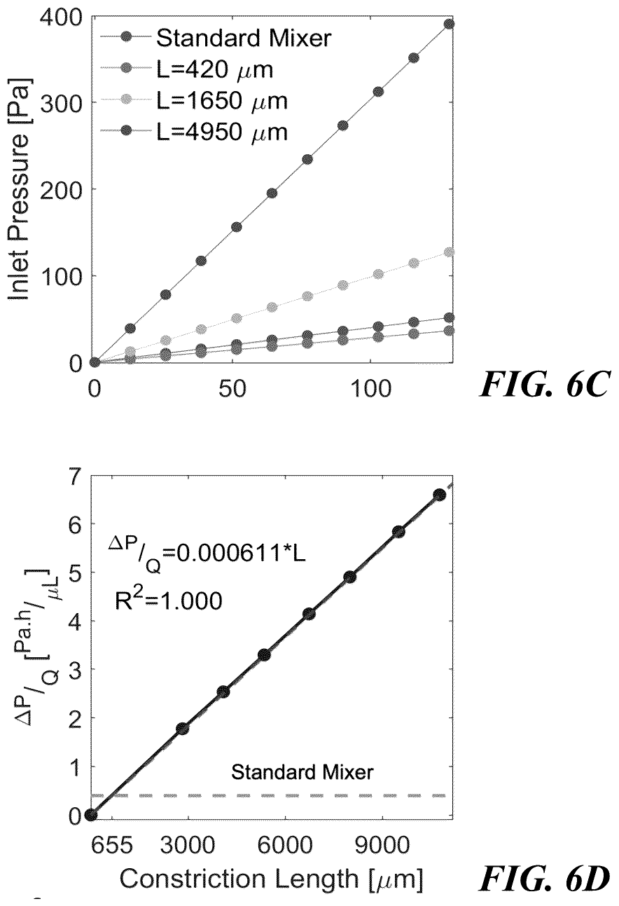

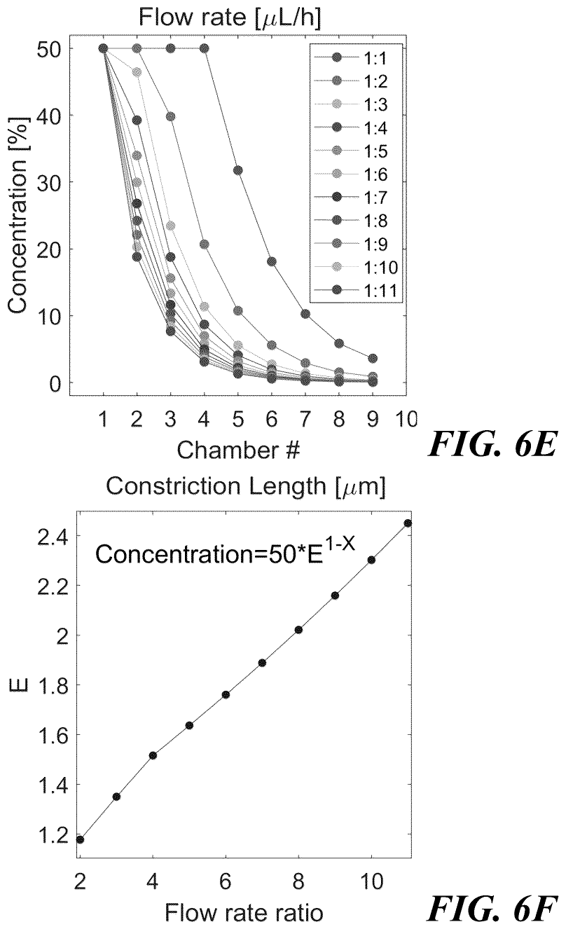

To find the resistance and flow within the mixer R_{M} (FIG. 6A):

$${R}_{{S}_{i + 1}} {q}_{s} = {R}_{{S}_{i}} {q}_{s} + {R}_{M} {Q}_{M} \qquad ( 1 )$$

where R is the resistance, M is the mixer, q is the flow rate, S is the side channels and i is the number of the loop. Then for each of the loops:

$${R}_{M} {q}_{M} = {R}_{{d}_{i}} {q}_{{sd}_{i}} \qquad ( 2 )$$

where q_{M}=1; q_{d}_{ᵢ}=1+0.5i; qₛ=0.5; and d is referred to as the resistances of the added sections. Substitution of the flow rates into Eqs. (1) and (2) provides:

$${R}_{{S}_{i + 1}} = {R}_{{S}_{i}} + 2 {R}_{M} \qquad ( 3 )$$

such that R_{d}_{ᵢ}(1+0.5i)=R_{M} and therefore

$${R}_{{d}_{i}} = \frac{2 {R}_{M}}{2 + i} .$$

Once the required resistances are determined, the length and width of each side channel is calculated to provide the desired resistance. A constriction (FIG. 5A, (iii)) in the width of a portion of the side channel increases hydraulic resistance which helps streamline the design compared to increasing the length of the serpentine.

Computational fluid dynamics (CFD) simulations were used to determine the hydraulic resistance of the constriction as a function of its length (FIGS. 6B-6D). The CFD simulations showed that a constriction of 40 μm×655 μm (width×length) has the same hydraulic resistance as a mixer (FIG. 6D). The details of the constrictions' length are depicted in FIGS. 10A-10D.

After the network layout and the dimensions of all resistors were determined, the concentration profile resulting from the network was examined by considering all the features in the chip. Since there are multiple features in the chip, a generic Matlab code that takes the pressure nodes (the black dots in FIG. 6A), the dimensions of the features that connect them, and the boundary conditions, then calculates the resulting flow rates in each feature and the concentrations of the drug at each node was written. The code is available on Github. Using this code, the concentration profile at various flow rate ratios between drug and diluent was calculated (FIG. 6E). Next, an exponential equation was fitted to the data; concentration (%)=50×E¹⁻ⁱ, to calculate E as the base of the power. Here i is the number of the loop (1-9). For 2-fold dilution, E needs to be 2 and the model suggests that the flow rate ratio of both inlets should be 7.8 (FIG. 6F).

The ladder microfluidic system is an improvement over previous designs (Nguyen et al., “Diffusion-Convection Hybrid Microfluidic Platform for Rapid Antibiotic Susceptibility Testing,” Anal. Chem. 93:5789-5796 (2021), which is hereby incorporated by reference in its entirety) as it allows for the generation of a broader range of concentrations with dilutions over two orders of magnitude. Further, the standardized concentration profile (2-fold dilution) ensures relevance and translatability of the results when interpreted with standard breakpoints set by national and international organizations such as the Clinical and Laboratory Standards Institute or the FDA.

### Example 3—Principle of Operation

The operation of the ladder microfluidic system follows a previously established protocol (Nguyen et al., “Diffusion-Convection Hybrid Microfluidic Platform for Rapid Antibiotic Susceptibility Testing,” Anal. Chem. 93:5789-5796 (2021); Azizi et al., “Nanoliter-Sized Microchamber/Microarray Microfluidic Platform for Antibiotic Susceptibility Testing,” Anal. Chem. 90:14137-14144 (2018); Azizi et al., “Gradient-Based Microfluidic Platform for One Single Rapid Antimicrobial Susceptibility Testing,” ACS Sens 6:1560-1571 (2021); Azizi et al., “Biological Small-Molecule Assays Using Gradient-Based Microfluidics,” Biosens. Bioelectron. 178:113038 (2021); and Azizi et al., “Antimicrobial Susceptibility Testing in a Rapid Single Test via an Egg-Like Multivolume Microchamber-Based Microfluidic Platform,” ACS Appl. Mater. Interfaces 13:19581-19592 (2021), each of which is hereby incorporated by reference in its entirety). The standardized cation-adjusted Mueller Hinton broth was used to prepare all the solutions, with 2 v/v % PrestoBlue added as a cell metabolism indicator. Loading of the platform consists of the following three steps: (1) a bacterial suspension was loaded into the device using a syringe (FIG. 5B, i); (2) an antibiotic solution in culture media and clean culture medium were loaded into the device through two different ports using syringe pumps to generate a concentration profile in the main channel network. This action washes away the bacteria in the main channel but leaves those inside the dead-end microchambers (FIG. 5B, ii). The antibiotic diffuses into the microchambers according to a drug specific loading time, after which, (3) the system is loaded with biological grade mineral oil to isolate the microchambers (FIG. 5B, iii). Once the microchambers are sealed off, the on-chip water bath is loaded with water, then the device is incubated at 37° C. for 4-5 hours (FIG. 11).

Four bacterial species representing pathogens relevant to canine UTI were selected: Escherichia coli (EC; n=14), Proteus mirabilis (PRM; n=8), Staphylococcus pseudintermedius (SP; n=5), Enterococcus faecalis (EF; n=3). Suspensions of these bacteria were prepared to be used in the platform at ˜3×10⁶ CFU mL⁻¹, which is slightly higher than the standard method which calls for concentrations around 5×10⁵ to 1×10⁶ CFU mL⁻¹. It was found that this concentration improved sample dispersions in all the microchambers without changing the resulting MICs. The result interpretation protocol was followed as previously established based on the difference in fluorescent intensities of resazurin as a cell metabolic indicator (Nguyen et al., “Diffusion-Convection Hybrid Microfluidic Platform for Rapid Antibiotic Susceptibility Testing,” Anal. Chem. 93:5789-5796 (2021); Azizi et al., “Nanoliter-Sized Microchamber/Microarray Microfluidic Platform for Antibiotic Susceptibility Testing,” Anal. Chem. 90:14137-14144 (2018); Azizi et al., “Gradient-Based Microfluidic Platform for One Single Rapid Antimicrobial Susceptibility Testing,” ACS Sens 6:1560-1571 (2021); Azizi et al., “Biological Small-Molecule Assays Using Gradient-Based Microfluidics,” Biosens. Bioelectron. 178:113038 (2021); and Azizi et al., “Antimicrobial Susceptibility Testing in a Rapid Single Test via an Egg-Like Multivolume Microchamber-Based Microfluidic Platform,” ACS Appl. Mater. Interfaces 13:19581-19592 (2021), each of which is hereby incorporated by reference in its entirety). It was hypothesized that there are three patterns of bacterial growth in the ladder microfluidic system: finding the minimum inhibitory concentration (MIC) (FIG. 5C, i), determining antibiotic resistance (or the MIC is higher than the maximum tested concentration, FIG. 5C, ii), or observing no growth due to a low number of bacteria or an invalid test (FIG. 5C, iii). The latter two scenarios can be assessed because a positive growth control was included in the system to validate the results.

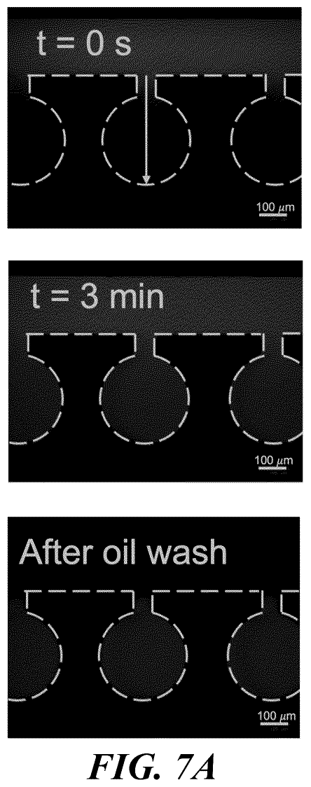

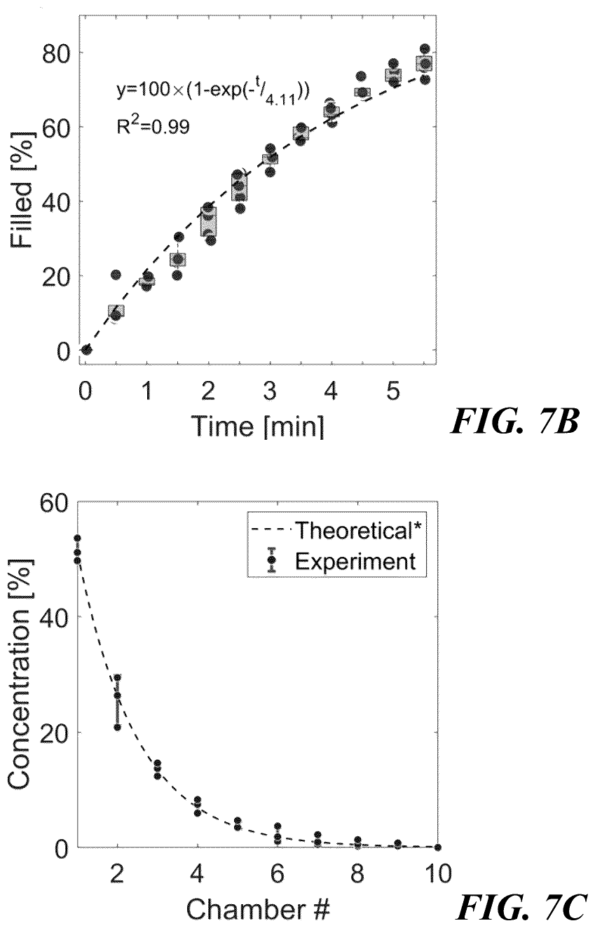

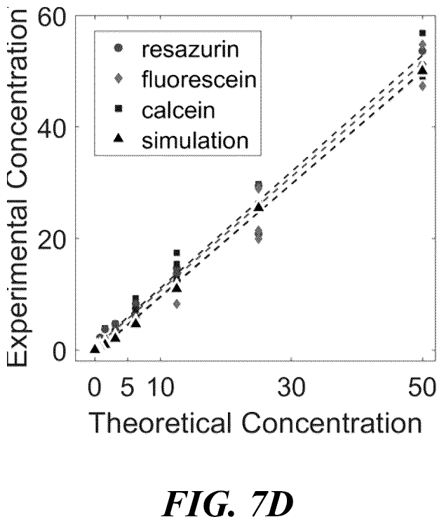

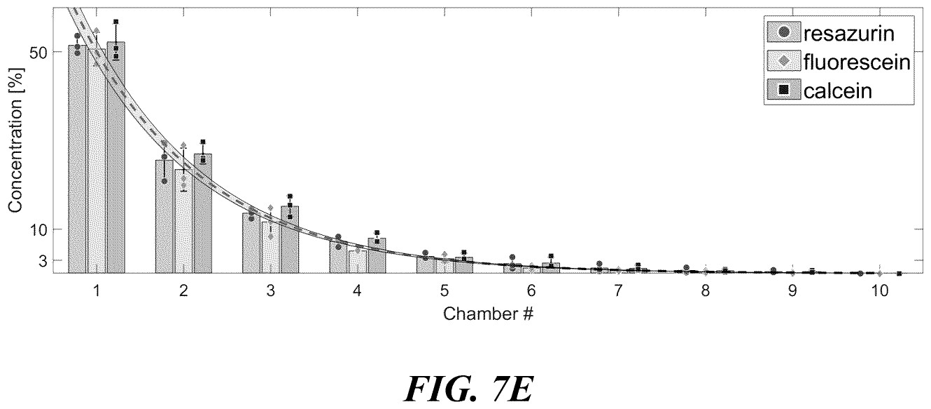

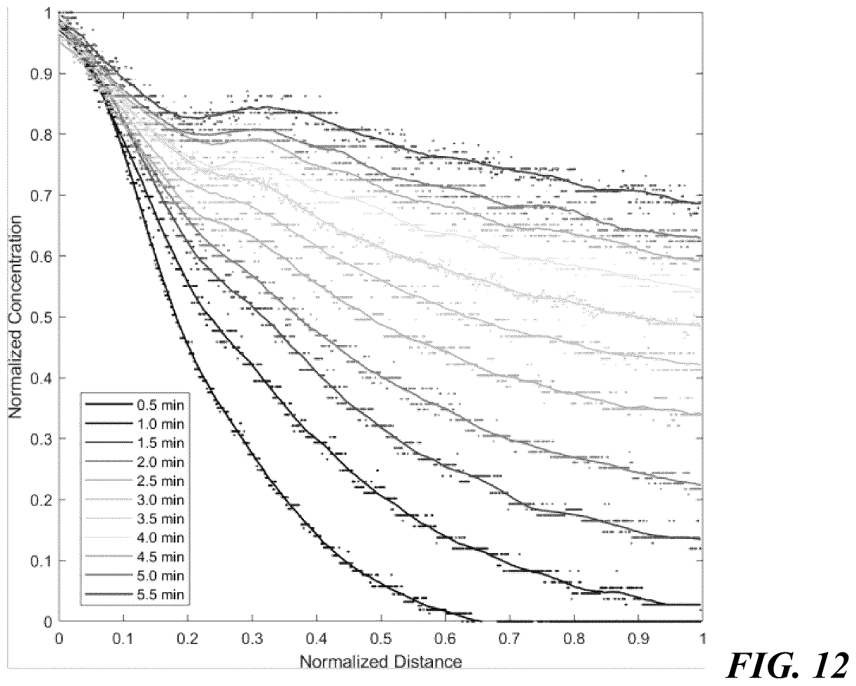

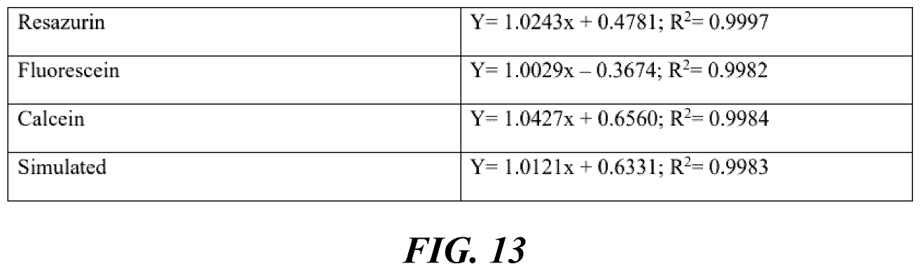

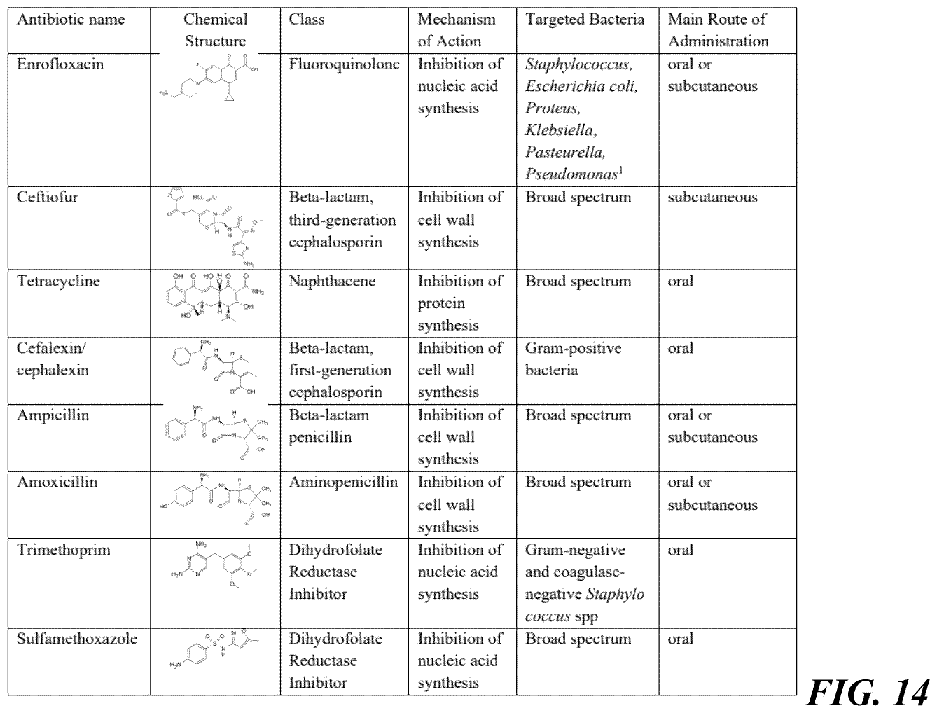

During the incubation period, it was we observed that the background fluorescence of the resazurin increased, however, the fluorescent intensity indicating bacterial metabolism remained distinguishable. Due to differences in growth rate and doubling times between the four species, the signal intensities were different among the Gram-negative and Gram-positive samples. More specifically, Gram-negative bacteria EC and PRM doubled much faster than the other Gram-positive bacteria and therefore had higher overall fluorescent intensities. However, it was observed that after the 4-5 hour incubation period, the changes in fluorescent intensities were sufficient to determine the MICs for all species. For most of the samples, especially the EC and PRM samples, results could be read after 4 hours and as early as 3.5 hours. For some of the Gram-positive samples, however, the signal took longer to develop due to slow growth rate, and was given an additional 30 minutes to 1 hour of incubation time. Prolonged incubation, up to 5 hours, of the Gram-negative bacteria did not change the MICs. Among the three steps, bacterial loading, antibiotic loading and microchamber sealing, only the loading time in step 2 (FIG. 7A) changes depending on which antibiotic is being tested. Characterizing the loading of antibiotics into the microchambers and resulting concentration profile were most important in determining the operational procedure of this system. First, the diffusion kinetics of resazurin into the microchambers was examined (FIG. 12). The resazurin molecules diffuse into the side channel (x₁=0−0.2) then into the main body of the microchamber (x₂=0.2−1). The concentration of resazurin increased in both regions. At t=3 minutes, the average concentration inside the microchamber (average area under the curve of x₂) was roughly 50% of the C₀ in the main channel and was assigned as the specific loading time for resazurin (FIG. 7B). The 50% cut-off was chosen for ease of antibiotic preparation, and to reduce the required loading time; resazurin did not reach saturation in the microchamber for almost 7 minutes. The resazurin concentrations in the ten microchambers form an exponential gradient (FIG. 7C). The concentration at microchamber 2 has the highest error, which corresponds with the oscillations occasionally observed at the first node of some devices due to differences in the fabrication process. The oscillation, and its standard deviation, however, was reduced as the flow regime became more stable in subsequent loops. Loading time is dependent on the molecular size, therefore, three dyes with different sizes were chosen to estimate how antibiotics with comparable sizes will diffuse. A linear relationship between loading time (T) and molar volume (V) such that (T₁*V₁=T₂*V₂) was previously demonstrated (Azizi et al., “Gradient-Based Microfluidic Platform for One Single Rapid Antimicrobial Susceptibility Testing,” ACS Sens 6:1560-1571 (2021) and Azizi et al., “Antimicrobial Susceptibility Testing in a Rapid Single Test via an Egg-Like Multivolume Microchamber-Based Microfluidic Platform,” ACS Appl. Mater. Interfaces 13:19581-19592 (2021), each of which is hereby incorporated by reference in its entirety). Using this, the loading time for other fluorescent dyes, fluorescein (208±4 cm³ mole⁻¹) and Calcein (356±5 cm³ mole⁻¹), was estimated based on the loading time of resazurin (145±7 cm³ mole⁻¹) and the concentration profiles of each molecule was measured under the respective loading time. A linear correlation between the concentration profiles of these three molecules and the theoretical 2-fold dilution concentration profile showed high accuracy with <5% error (FIG. 7D, FIG. 13). Individual concentrations of these profiles also follow the same trend (FIG. 7E). Based on this operating principle, the loading time for most relevant antibiotics would be equal or <10 min for each. Specifically, the loading times for the antibiotics used in the ASTs in this work ranged from 4 minutes 47 seconds to 5 minutes 57 seconds. However, the specific loading times only apply if a single antibiotic is used. When drug combinations that contain molecules with different loading rates, such as Amoxicillin/Clavulanic acid and Trimethoprim/Sulfamethoxazole are used, a mismatch in concentration gradients will result if only one of the two antibiotic loading times is considered. In this case, the solutions of antibiotic mixtures were loaded for 10 minutes to ensure saturation of all molecules in the microchambers.

### Example 4—Performing AST With Clinical Isolates

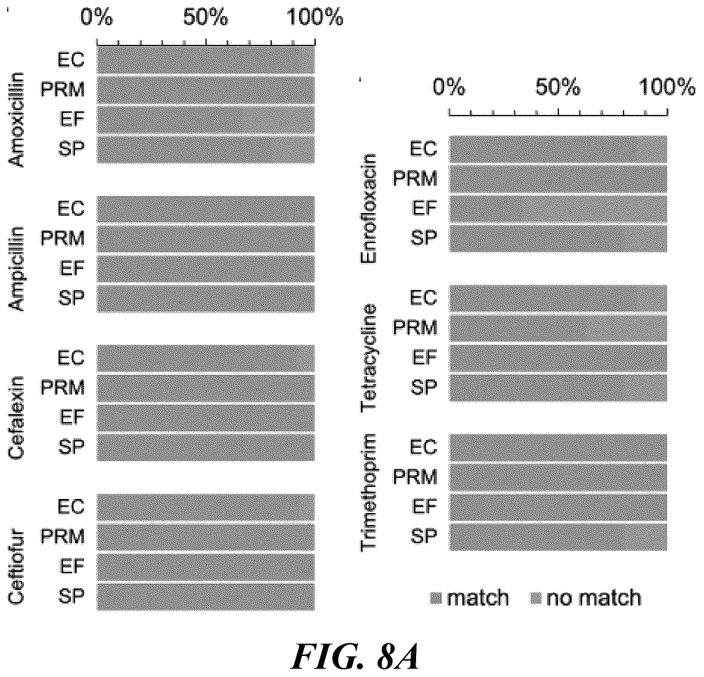

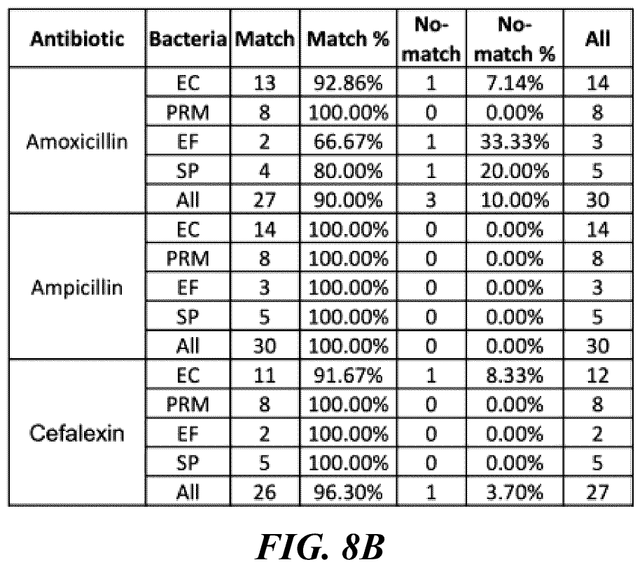

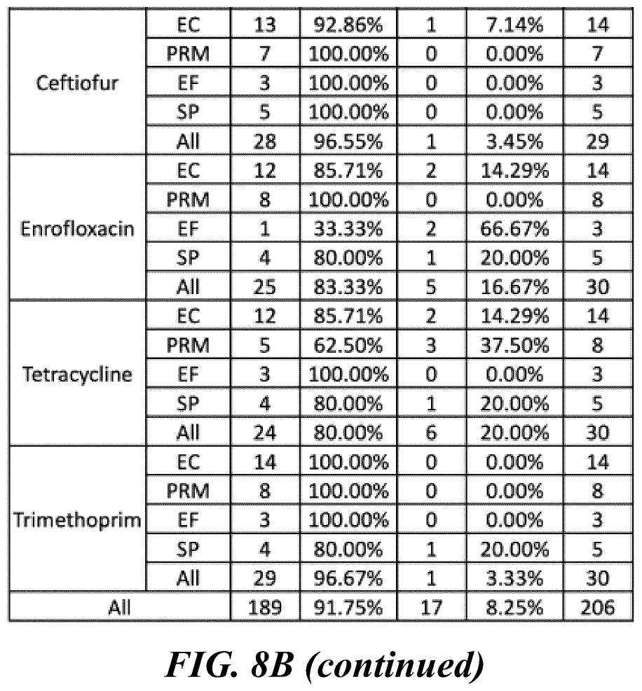

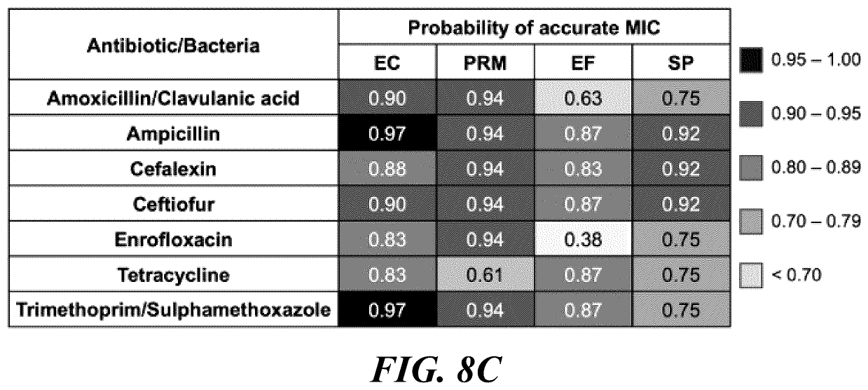

The performance characteristics of the ladder microfluidic system in determining MICs were examined with bacterial isolates from canine urinary tract infection samples which were submitted to the Cornell Animal Health Diagnostic Center (AHDC) for AST (FIG. 8A). The four most prevalent infectious organisms in canine UTI cultured at the facility between 2007-2017 are: Escherichia coli (EC), Proteus mirabilis (PRM), Enterococcus faecalis (EF), and Staphylococcus pseudintermedius (SP). These four bacterial species accounted for 66.7% of all cases. Thirty (30) bacterial isolates were tested in total. The representations of the four bacteria in the sample reflects their clinical distributions: EC (n=14), PRM (n=8), SP (n=5), and EF (n=3). AST of these organisms was carried out against the seven antibiotics listed on the Sensititre Veterinary UTI plate (SVU), which is used by the AHDC to analyze these samples: Enrofloxacin, Ceftiofur, Tetracycline, Cefalexin, Ampicillin, and two antibiotic combinations Amoxicillin/Clavulanic acid, and Trimethoprim/Sulfamethoxazole (FIG. 13).

For the purposes of result comparison, the range of concentration and the combination ratios for these antibiotics follow those on the SVU plate. To compare the MICs obtained by AST using the ladder microfluidic system and using the SVU, FDA guidance for characterizing the performance of new AST systems was followed and the data was interpreted based on the number of agreements. First, the on-chip MIC and the MIC obtained from the SVU plate as carried out by the AHDC was paired for each bacteria/antibiotic combination. An agreement designation (match) was assigned if the on-chip MIC was within ±one 2-fold dilution. For many instances, the MIC determined by the SVU was in the form of a range, for example, many of the MICs for AST with Trimethoprim/Sulfamethoxazole were ≤2 μg mL⁻¹. For these pairs, a match was confirmed if the on-chip MIC fulfilled the condition of the SVU plate MIC (for example, on-chip MIC=0.5 μg/mL was a match for SVU MIC ≤2 μg mL⁻¹). There were four occasions where data from the SVU (three for Cephalexin and one for Ceftiofur) was not available, so these were excluded from the statistical evaluation.

In total, 206 bacterial samples were evaluated for MIC on both the ladder microfluidic system and the gold standard SVU plates. An overall 91.75% accuracy of determining the MIC between the two methods was observed; 189 samples matched between the two platforms. Further, the matching rate for each individual bacteria strain was calculated as the number of samples that matched (m) divided by the total number of bacteria samples of that type (n). Among the four bacteria, EC and PRM had the highest matching rate at 92.71% ( 89/96) and 94.54% ( 52/55), respectively, and SP and EF had the lowest matching rate at 88.57% ( 31/35) and 85.00% ( 17/20), respectively.

The data was also examined from the perspective of individual bacterial species, antibiotics, and their combinations (FIG. 8B). When examining the accuracy with respect to each antibiotic, it was found that on-chip AST with Ampicillin had the highest MIC accuracy at 100.0%, followed by Cefalexin (96.30%), Ceftiofur (96.55%), Trimethoprim/Sulfamethoxazole (96.67%), and Amoxicillin/clavulanic acid (90.00%). The worst performing antibiotics in terms of aligning with the results from SVU were Enrofloxacin (83.33%) and Tetracycline (80.00%). In the analysis for the probability of correctly obtaining a MIC for each bacteria/antibiotic combination, a similar pattern where EC and PRM had the highest probability, as high as 97% for EC (FIG. 8C), was observed. The lowest probability was observed with the EF/Enrofloxacin combination, which is likely inconclusive due to sample size (n=3). Overall, a statistically significant difference in the probability of achieving an accurate MIC using the ladder microfluidic method in comparison to the SVU was not observed.

To further understand the potential sources of errors leading to disagreements in some of the bacteria/antibiotic groups, those samples which had more than three disagreements were repeated. While improvement in accuracy with some samples was observed after repeating, suggesting random handling error in the first experiment, most of them had the same MICs. The same cannot be said for the original SVU data obtained from AHDC, which was conducted once. This observation indicates that the exemplified method is reliable and repeatable, and the mismatch between the MICs could be due to errors from the SVU as run by the AHDC.

### Example 5—Direct-From-Sample AST With Spiked and Clinical Samples

While the time-to-result of the AST of clinical isolates was reduced, whether the overall time required for obtaining a susceptibility result can further be reduced for some urine samples, up to 24 hours, by bypassing the bacteria isolation step was next investigated (Baltekin et al., “Antibiotic Susceptibility Testing in less than 30 min Using Direct Single-Cell Imaging,” Proc. Natl. Acad. Sci. USA 114:9170-9175 (2017); Li et al., “Adaptable Microfluidic System for Single-Cell Pathogen Classification and Antimicrobial Susceptibility Testing,” Proc. Natl. Acad. Sci. USA 116, 10270-10279 (2019); Avesar et al., “Nanoliter Cell Culture Array with Tunable Chemical Gradients,” Anal. Chem. 90:7480-7488 (2018); Mo et al., “Rapid Antimicrobial Susceptibility Testing of Patient Urine Samples Using Large Volume Free-Solution Light Scattering Microscopy,” Anal. Chem. 91:10164-10171 (2019); Sabhachandani et al., “Integrated Microfluidic Platform for Rapid Antimicrobial Susceptibility Testing and Bacterial Growth Analysis Using Bead-Based Biosensor via Fluorescence Imaging,” Microchim. Acta 184:4619-4628 (2017); and Dong & Zhao, “Rapid Identification and Susceptibility Testing of Uropathogenic Microbes via Immunosorbent ATP-Bioluminescence Assay on a Microfluidic Simulator for Antibiotic Therapy,” Anal. Chem. 87:2410-2418 (2015), which are hereby incorporated by reference in their entirety). Thus, the ability of the presently claimed device to conduct AST directly with urine samples was evaluated. The study was conducted with seven urine samples that were cultured positive for the presence of a single unknown bacteria. Because these are canine urine samples, the volumes were limited, ranging from 2-3 mL. Each urine sample (2 mL) was first centrifuged at 13,000 g for 1 minute, and the resulting pellet resuspended in 1 mL of fresh MHB. Afterward, the suspension was filtered through a 5 μm syringe filter to remove any debris or eukaryotic cells. It was found that this sequence of bacterial collection was the most consistently effective for the limited volume of samples. The first step collects and concentrates all the bacteria and cells or debris inside the sample, while the second step isolates the targeted bacteria. This centrifuge-filter sequence leads to a decrease in bacteria loss as the larger pellet from the first spinning step helps in recovering bacterial cells. It was found that samples containing >10⁵ CFU mL⁻¹ gave consistent results, and thus excluded the diagnosis low-end of 10⁴−10⁵ CFU mL⁻¹. This limit is considered as the limit of detection of the method when testing directly from urine, however, most of the samples received at the facility are above 10⁵ CFU mL⁻¹. Finally, the isolated bacteria were preincubated for 2 hours to bring the cells out of lag phase before conducting AST. Similar to the original method with bacterial isolates, the MICs of these bacteria from clinical samples was determined in 4-5 hours. It is believed that the preincubation period is necessary when testing bacteria directly isolated from stored patient urine samples, such as the samples in this study, which were refrigerated around 24-36 hours before they were received for testing. This period appears to bring the bacteria out of the lag phase induced by refrigeration.

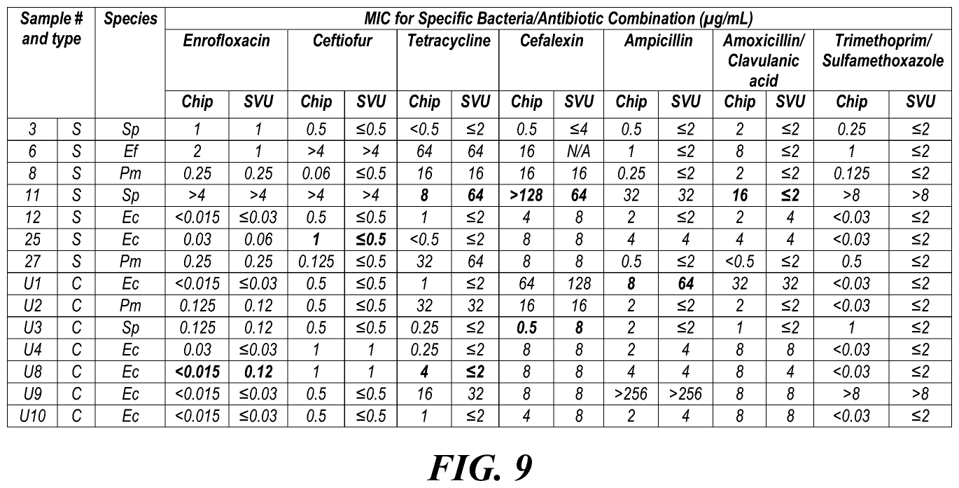

Some culture negative samples were tested and the lack of false positives was confirmed by observing no growth-indicative changes in the fluorescent intensity in the positive growth chambers which did not get exposed to antibiotics. Additionally, seven clinical isolates from the previously analyzed samples were spiked into pooled canine culture negative urine, and the same isolation/pre-enrichment process was carried out before performing AST. The same overall rate of agreement between the clinical and spiked samples was observed (91.84%) (FIG. 9), which is very similar to the rate observed in our study with the clinical isolates (91.75%) (FIG. 8B). Thus, it was concluded that the ladder microfluidic platform can perform AST for either bacterial isolates or bacteria directly collected from urine samples. A consistent discrepancy between MICs obtained using the presently disclosed invention and those obtained using the SVU plate as performed in the clinic was not observed, except for spiked sample 11 which had three discrepancies. Among those three discrepancies, only one (versus cephalexin) was consistent with the result from the first bacterial isolates analysis, leading us to believe that the other discrepancies were most likely the result of random errors such as operation or differences in device fabrication.

When selecting the culture positive urine samples, any sampling method that would generate a liquid volume sample were not excluded (swab samples were excluded), or type of collection tube, some of which contain additives to stabilize bacterial count. Comparing the results from all the urine samples suggests that the sample collection method does not have a effect on the performance of the method in determining MIC. These results collectively support direct-from-sample AST using the ladder microfluidic system.

### Discussion of Examples 1-5

Examples 1-5 describe the design, optimization, and use of a microfluidic system according to the present disclosure that performs phenotypic AST; incorporates standardized, translatable testing parameters; and tests both isolated bacteria from culture plates or bacteria isolated directly from urine samples. The system utilizes a ladder shaped network to generate a serially 2-fold diluted concentration gradient that follows current standardized methods and regulations and is tunable to accommodate a broad number of clinically relevant antibiotics and antibiotic combinations. The total preparation time, which includes bacteria loading, antibiotic loading, and oil loading, can be as fast as 10 minutes when testing with a single antibiotic and no >15 minutes for an antibiotic combination. An important consideration of the ladder microfluidic system is the two-fold serial dilution of the antibiotics on the microscale and within a small space. Although exponential concentration gradients have been generated in microscale, by adapting traditional gradient generators such as the Christmas tree (Chen et al., “A Review on Microfluidics Manipulation of the Extracellular Chemical Microenvironment and its Emerging Application to Cell Analysis,” Anal. Chim. Acta 1125:94-113 (2020), which is hereby incorporated by reference in its entirety), or H-filter (Selimović et al., “Generating Nonlinear Concentration Gradients in Microfluidic Devices for Cell Studies,” Anal. Chem. 83:2020-2028 (2011), which is hereby incorporated by reference in its entirety), these designs tend to be larger in size. Controlling the fluidic flow in a ladder-shape grid results in a simpler and smaller device, which is also easier to handle. This ladder microfluidic system is the first, to date, to use circuit logic to generate a concentration gradient in microfluidics.

Rapid AST can be performed on both bacterial isolates which is the standard type of sample for AST, or on clinical urine samples that contain the disease-causing pathogen. Compared with standard AST which requires 18-24 hours, the time-to-result using this microfluidic platform is shorter; results can be obtained within a single working day. The Examples of the present disclosure demonstrate that the presently disclosed method is >90% effective at determining the MIC of the most common UTI pathogens in canines against disease relevant antibiotics, when examined with 30 bacterial isolates from clinical samples. Specifically, the method was able to determine the MICs of the Gram-negative bacteria E. coli and P. mirabilis accurately up to 92.71% and 94.54%, respectively. The rate of accuracy for the other two Gram-positive bacteria, S. pseudintermedius and E. faecalis that we tested were slightly lower, at 88.57% and 85.00%, respectively, but no statistical significance was found. Further, the presently disclosed method is capable of testing bacteria recovered directly from clinical samples, eliminating the need to culture the pathogen for 12-48 hours. Because of the small dimensions at the microscale, the presently disclosed method does require a higher inoculum than the standardized method to minimize sampling effects. This requirement is not an issue when testing bacterial isolates plated on culture plates, but poses a challenge when testing clinical samples especially at the lower UTI diagnostic concentration 10⁴-10⁵ CFU mL⁻¹. Bacteria losses during the recovery process is the biggest contributing factor to this challenge. Therefore, a better recovery method is needed, or testing of isolated and enriched bacteria would be more appropriate for these samples. The method is effective for urine with higher bacteria concentration, which was most of the UTI diagnosed samples.

In a preliminary experiment, bacterial loads from 10⁴ to 10⁸ CFU mL⁻¹ were evaluated. A higher frequency of false negative growth results were observed in the 10⁴-10⁵ range, most likely due to sampling error because of the small volume of the microchambers. An increase in MIC with the 10⁷-10⁸ CFU L⁻¹ sample was observed, because there were too many bacterial cells. Out of the 189 clinical MICs from isolates, 64 of them were discrete values (not a <= or > range). Among them, 19 had higher MIC, 28 had lower MIC, and 17 had the same MIC as the standard method. In total, 9 out of the 64 received the no-match designation. This trend was improved in the later experiments with the direct from urine samples where we had 39 discrete MICs, with 2 higher MICs, 11 lower MICs, and 23 exact MICs, with an overall 5 no-match designations. This improvement is most likely due to better familiarity with the operation of the platform and assay. Interestingly, there was an increase in the frequency of MICs that were lower than the standard method, which was not expected with a higher than standard bacterial load. However, most of these decreases in MIC are within the acceptable range for method comparison. Furthermore, the PDMS material of the disclosed device is known to absorb some small molecules, especially hydrophobic molecules (Regehr et al., “Biological Implications of Polydimethylsiloxane-Based Microfluidic Cell Culture,” Lab. Chip 9:2132-2139 (2009), which is hereby incorporated by reference in its entirety), however the data presented herein neither strongly supports, nor fully rejects, the effect of adsorption on the MICs.

The performance of the system was characterized on urine samples containing a single organism and samples with multiple organisms were excluded. This is a limitation for many phenotypic AST methods aiming to test directly from samples where the bacteria species is unknown. Depending on the sample collection process, urine samples can be contaminated with commensal bacteria, and therefore it is difficult to confidently identify the presence of the pathogen(s). Further, false classification of antibiotic resistance might occur if one or more of the contaminants is resistant to an antibiotic, resulting in erroneous exclusion of a potentially effective therapy. Conducting AST on multi-organism samples will require more complex result interpretation and may need the judgment of medical professionals for final diagnostic determinations. For these reasons, multi-organism samples were not included in the scope of this study.

The characterization studies focused on the four most common pathogens of UTIs at a veterinary diagnostic center, and the results from those bacteria suggests that the method could be extended to additional species of Gram-negative and Gram-positive UTI pathogens. The usability of the disclosed method to test bacterial isolates in place of the current SVU plate was demonstrated, as well as the feasibility to test bacteria retrieved directly from samples at a clinically relevant concentration. UTIs are among the most common bacterial infections dealt with at a clinical microbiology laboratory. While bacteria speciation is required for final susceptibility determination, the disclosed method can perform AST without prior information on the bacteria and interpreted after identification of bacteria is confirmed.

A rapid AST method would improve patient outcome and streamline clinical laboratory workflow by 18 hpurs if tested with identified bacterial isolates. Using the rapid urine test, AST can be conducted as soon as a sample is suspected to contain a UTI pathogen, while other identification methods such as MALDI-TOF are carried out simultaneously to identify bacteria, potentially reducing the time to result up to 2 days. Rapid AST methods can be used in place of, or in complement to, the SVU plate method. A Rapid AST test that could be read within a day would allow veterinarians and medical professionals to provide more targeted initial prescription during early diagnosis. This would reduce the use of broad-spectrum antibiotics and decrease the potential for antibiotic resistance. With improvement in testing time and adherence to industry standards, it is believed that this platform can be used at various stages in the clinical workflow, and thus provides another tool for combating antibiotic resistance without compromising patient health or increasing costs.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A microfluidic circuit comprising:
a drug inlet port;
an outlet port;
a drug inlet main channel fluidically connecting the drug inlet port and the outlet port, wherein said drug inlet main channel comprises (i) a plurality of serpentine mixers and (ii) a plurality of dead-end first microchamber sets;
a negative inlet port;
a negative inlet main channel fluidically connecting the negative inlet port and the drug inlet main channel;
a plurality (n) of ladder channels, wherein each of the plurality (n) of the ladder channels is fluidically connected to both the drug inlet main channel and the negative inlet main channel; and
an outlet channel fluidically connected to the drug inlet main channel between the drug inlet port and the outlet port.

2. The microfluidic circuit according to claim 1, wherein the drug inlet main channel and the negative inlet main channel are fixedly positioned parallel to each other.

3. The microfluidic circuit according to claim 1, wherein each of the ladder channels is positioned perpendicular to each of the main channels.

4. The microfluidic circuit according to claim 3, wherein the ladder channels are fixedly positioned parallel to each other between the drug inlet main channel and the negative inlet main channel.

5. The microfluidic circuit according to claim 1, wherein n−1 of the plurality (n) of ladder channels each comprises a hydraulic resistor pinch.

6. The microfluidic circuit according to claim 5, wherein the ladder channel positioned most proximate to the drug inlet port does not possess a hydraulic resistor pinch.

7. The microfluidic circuit according to claim 5, wherein the hydraulic resistor pinch in each of the ladder channels is of a different length than the other ladder channels.

8. The microfluidic circuit according to claim 7, wherein the length of each hydraulic resistor pinch increases in the direction of the drug inlet port to the outlet port.

9. The microfluidic circuit according to claim 1, wherein each of the microchambers in the plurality of dead-end first microchamber sets is individually fluidically connected to the drug inlet main channel by a side channel.

10. The microfluidic circuit according to claim 1, wherein a pair of ladder channels in sequence between the drug inlet main channel and the negative inlet main channel comprises a subunit of the microfluidic circuit.

11. The microfluidic circuit according to claim 10, wherein each subunit comprises only one of the dead-end first microchamber sets.

12. The microfluidic circuit according to claim 10, wherein at least one of the subunits comprises a plurality of dead-end second microchamber sets.

13. The microfluidic circuit according to claim 12, wherein each of the dead-end second microchambers of the dead-end second microchamber sets is individually fluidically connected to the negative inlet main channel by a side channel.

14. The microfluidic circuit according to claim 12, wherein the plurality of dead-end second microchamber sets is comprised of three distinct microchambers.

15. The microfluidic circuit according to claim 1, wherein the plurality of dead-end first microchamber sets is comprised of three distinct microchambers.

16. The microfluidic circuit according to claim 1, wherein the outlet channel is positioned parallel to the drug inlet main channel.

17. The microfluidic circuit according to claim 16, wherein the outlet channel is fluidically connected to the drug inlet main channel by one or more outlet side channels.

18. The microfluidic circuit according to claim 17, wherein only a single outlet side channel is positioned in the drug inlet main channel between proximate pairs of the ladder channels.

19. The microfluidic circuit according to claim 1, wherein the outlet channel comprises a plurality of serpentine mixers.

20. The microfluidic circuit according to claim 19, wherein only a single serpentine mixer is positioned in the outlet channel between proximate pairs of the ladder channels.

21. A microfluidic device comprising:
a support layer;
a substrate layer disposed on the support layer; and
a microfluidic circuit of claim 1, wherein the microfluidic circuit is disposed within the substrate layer.

22-42. (canceled)

43. A method for performing an assay, said method comprising:
loading a test solution into the microfluidic device of claim 1;
loading a culture medium containing an antibiotic solution into the drug inlet port;
loading a culture medium into the negative medium inlet port;
washing the device with oil to isolate each of the plurality of microchambers; and
detecting a fluorescent signal in each of the plurality of microchambers.

44-46. (canceled)
